SEQUENCE LISTING (16 sequences)

SEQ ID NO: 1 - protein, 7 aa, Artificial Sequence
  anti-CD38 single-domain antibody Nb2F8 CDR1
YTDSDYI

SEQ ID NO: 2 - protein, 10 aa, Artificial Sequence
  anti-CD38 single-domain antibody Nb2F8 CDR2
TIYIGGTYIH

SEQ ID NO: 3 - protein, 18 aa, Artificial Sequence
  anti-CD38 single-domain antibody Nb2F8 CDR3
AATKWRPFISTRAAEYNY

SEQ ID NO: 4 - protein, 125 aa, Artificial Sequence
  anti-CD38 single-domain antibody Nb2F8
QVQLVESGGGSVQAGGSLRLSCAASGYTDSDYIMAWFRQAPGKEREVVATIYIGGTYIHYADSVKGRFTISRDNAENTVYLQMNNLKPEDTAMYYCAATKWRPFISTRAAEYNYWGQGTLVTVSS

SEQ ID NO: 5 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 6 - protein, 26 aa, Artificial Sequence
  anti-CD38 single-domain antibody Nb2F8 FR1
QVQLVESGGGSVQAGGSLRLSCAASG

SEQ ID NO: 7 - protein, 16 aa, Artificial Sequence
  anti-CD38 single-domain antibody Nb2F8 FR2
MAWFRQAPGKEREVVA

SEQ ID NO: 8 - protein, 37 aa, Artificial Sequence
  anti-CD38 single-domain antibody Nb2F8 FR3
YADSVKGRFTISRDNAENTVYLQMNNLKPEDTAMYYC

SEQ ID NO: 9 - protein, 11 aa, Artificial Sequence
  anti-CD38 single-domain antibody Nb2F8 FR4
WGQGTLVTVSS

SEQ ID NO: 10 - protein, 22 aa, Artificial Sequence
  FL linker
EFPKSSTPPGSSGGAPGSGSGS

SEQ ID NO: 11 - DNA, 66 bp, Artificial Sequence
  FL linker
gaatttccgaaatcgagcaccccgccgggcagcagcggcggcgcgccgggcagcggcagcggcagc

SEQ ID NO: 12 - protein, 6 aa, Artificial Sequence
  Sortase A recognition motif
LPETGG

SEQ ID NO: 13 - DNA, 18 bp, Artificial Sequence
  Sortase A recognition motif
ctgccggaaaccggcggc

SEQ ID NO: 14 - protein, 5 aa, Artificial Sequence
  amino acid portion of PNA1 hybridisation probe
GGGSS

SEQ ID NO: 15 - DNA, 15 bp, Artificial Sequence
  polypeptide nucleic acid (PNA) portion of PNA1 hybridisation probe
agtctggatgtagtc

SEQ ID NO: 16 - DNA, 15 bp, Artificial Sequence
  polypeptide nucleic acid (PNA) portion of PNA2 hybridisation probe
gactacatccagact

(19) United States
(12) Patent Application Publication
(10) Pub. No.: US 2023/0190968 A1
(43) Pub. Date: Jun. 22, 2023

(54) ANTI-CD38 SINGLE-DOMAIN ANTIBODIES IN DISEASE MONITORING AND TREATMENT

(71) Applicants: UNIVERSITÉ DE LIÈGE, Liège (BE); CENTRE HOSPITALIER UNIVERSITAIRE DE LIÈGE, Liège (BE); VRIJE UNIVERSITEIT BRUSSEL, Brussels (BE)

(72) Inventors: Jo CAERS, Liège (BE); Elodie DURAY, Liège (BE); Mireille DUMOULIN, Liège (BE); Matthias D'HUYVETTER, Brussels (BE); Amelie ERIKSSON KARLSTRÖM, Bromma (SE)

(73) Assignees: UNIVERSITÉ DE LIÈGE, Liège (BE); CENTRE HOSPITALIER UNIVERSITAIRE DE LIÈGE, Liège (BE); VRIJE UNIVERSITEIT BRUSSEL, Brussels (BE); KTH HOLDING AB, Stockholm (SE)

(21) Appl. No.: 17/925,559

(22) PCT Filed: May 17, 2021
(86) PCT No.: PCT/EP2021/063021

§ 371 Date: Nov. 15, 2022

(30) Foreign Application Priority Data
May 15, 2020 (EP) 20175032.0

(51) Int. Cl. A61K 51/10; C07K 16/28; A61P 35/00

(52) U.S. Cl. CPC A61K 51/1027; C07K 16/2896; A61K 51/1069; A61P 35/00; C07K 2317/569; C07K 2317/92; A61K 2039/505

(57) ABSTRACT

The present invention relates to medical imaging, disease monitoring and theranostic approaches in neoplastic diseases of certain anti-CD38 single-domain antibodies (sdAb).

### FIELD

The invention is broadly in the medical imaging, diagnostics and theranostics fields, and more particularly pertains to evaluating, monitoring and treatment of neoplastic diseases.

### BACKGROUND

The field of theranostics aims to develop more specific, individualised therapies for various diseases, and to combine diagnostic and therapeutic capabilities into a single pharmaceutical agent.

Recent advances in nanomaterials technology have prompted the development of different theranostic agents. For example, Nanobodies® (Nbs) are single-domain antigen-binding fragments that are derived from Camelidae heavy-chain antibodies and have emerged as a new targeting tool (De Meyer et al. Nanobody-based products as research and diagnostic tools. Trends Biotechnol 2014, vol. 32, 263-70). Compared to conventional antibodies, their small size leads to better tissue penetration, favourable pharmacological properties and the ability to recognise small, buried epitopes. Nbs specifically bind cellular targets with high affinity while unbound Nbs are rapidly cleared from non-targeted tissues. Certain Nbs have been developed as radiotracers for diagnostic imaging in animal models of cancer, inflammation and cardiovascular diseases (D'Huyvetter et al. Radiolabeled nanobodies as theranostic tools in targeted radionuclide therapy of cancer. Expert Opin Drug Deliv. 2014, vol. 11, 1939-54). Some Nbs have been coupled with toxins, chemotherapeutics, prodrug-activating enzymes or nanoparticles to provide for cancer therapeutics (Yu et al. Humanized CD7 nanobody-based immunotoxins exhibit promising anti-T-cell acute lymphoblastic leukemia potential. Int J Nanomedicine 2017, vol. 12, 1969-83; Fang et al. Structurally Defined alphaMHC-II Nanobody-Drug Conjugates: A Therapeutic and Imaging System for B-Cell Lymphoma. Angew Chem hit Ed Engl. 2016, vol. 55, 2416-20). Some authors have also discussed the option of using Nbs as vehicles for targeted radionuclide therapy (D'Huyvetter et al. 2014, supra; Dekempeneer et al. Targeted alpha therapy using short-lived alpha-particles and the promise of nanobodies as targeting vehicle. Expert Opin Biol Ther. 2016, vol. 16, 1035-47). The development of novel, effective theranostic agents is required for those cancers with a high unmet clinical need.

CN109232739 to Univ Peking Shenzen Graduate School, Li et al. Immuno-targeting the multifunctional CD38 using nanobody. Sci Rep. 2016, vol. 6, 27055, and An et al. Anti-Multiple Myeloma Activity of Nanobody-Based Anti-CD38 Chimeric Antigen Receptor T Cells. Mol Pharm. 2018, vol. 15, 4577-88, describe certain anti-CD38 nanobodies and their use in immunotoxin- or Chimeric Antigen Receptor T Cells (CAR-T)-based targeting strategies in cell or animal models of multiple myeloma, a hematological malignancy characterised by high expression of CD38.

### SUMMARY

The present invention is at least in part based on the unexpected finding that certain anti-CD38 single-domain antibodies (sdAb) display advantageous properties which render them especially useful in medical imaging, disease monitoring and theranostic approaches in neoplastic diseases. In particular, as evidenced in the experimental section of this specification which documents certain illustrative embodiments of the present invention, unlike daratumumab (trade name Darzalex®), the IgG1 human monoclonal anti-CD38 antibody currently used in clinic (de Weers et al. Daratumumab, a novel therapeutic human CD38 monoclonal antibody, induces killing of multiple myeloma and other hematological tumors. J Immunol. 2011, vol. 186(3), 1840-8), the present anti-CD38 sdAbs do not induce or induce only minimal internalisation of the CD38 antigen-antibody complex from the surface of the cell into its interior, and thereby also avoid or lessen the downregulation of CD38 expression on the cell membrane. Consequently, the expression of the CD38 antigen on the cell surface is largely preserved, which allows for further effective targeting of the antigen for imaging, monitoring and/or therapeutic purposes. Additionally, the present anti-CD38 sdAbs bind to CD38 epitopes other than the epitope recognised by daratumumab, and may display no interference or competition with daratumumab for CD38 binding. This opens an avenue to combination therapies, or combined imaging/diagnosis and therapy applications with said anti-CD38 sdAbs and daratumumab.

In view of these advantages, an aspect of the invention provides an anti-CD38 single-domain antibody directly or indirectly coupled to a second molecule for use in a method of diagnosis or monitoring a neoplastic disease in a subject, or for use in a method of treating a neoplastic disease in a subject, wherein the antibody comprises an amino acid sequence that comprises 3 complementary determining regions (CDR1 to CDR3);

wherein CDR1 is chosen from the group consisting of:
a) YTDSDYI (SEQ ID NO: 1),
b) Polypeptides that have at least 80% amino acid sequence identity with SEQ ID NO: 1,
c) Polypeptides that have 3, 2 or 1 amino acid difference with SEQ ID NO: 1,

wherein CDR2 is chosen from the group consisting of:
a) TIYIGGTYIH (SEQ ID NO: 2),
b) Polypeptides that have at least 80% amino acid sequence identity with SEQ ID NO: 2,
c) Polypeptides that have 3, 2 or 1 amino acid difference with SEQ ID NO: 2,

and wherein CDR3 is chosen from the group consisting of:
a) AATKWRPFISTRAAEYNY (SEQ ID NO: 3),
b) Polypeptides that have at least 80% amino acid sequence identity with SEQ ID NO: 3,
c) Polypeptides that have 3, 2 or 1 amino acid difference with SEQ ID NO: 3.

A related aspect provides a method for diagnosis or monitoring a neoplastic disease in a subject, the method comprising administering to the subject an anti-CD38 single-domain antibody directly or indirectly coupled to a second molecule, wherein the antibody comprises an amino acid sequence that comprises the 3 above-defined complementary determining regions CDR1 to CDR3.

A further aspect provides an anti-CD38 single-domain antibody directly or indirectly coupled to a second molecule, wherein the antibody comprises an amino acid sequence that comprises the 3 above-defined complementary determining regions CDR1 to CDR3.

A related aspect provides a method for diagnosis or monitoring and treating a neoplastic disease in a subject, the method comprising administering to the subject a therapeutically effective amount of anti-CD38 single-domain antibody directly or indirectly coupled to a second molecule, wherein the antibody comprises an amino acid sequence that comprises the 3 above-defined complementary determining regions CDR1 to CDR3.

Also provided is a method for treating a neoplastic disease in a subject, the method comprising administering to the subject a therapeutically effective amount of anti-CD38 single-domain antibody directly or indirectly coupled to a second molecule, wherein the antibody comprises an amino acid sequence that comprises the 3 above-defined complementary determining regions CDR1 to CDR3.

An additional aspect provides an imaging method for evaluating or monitoring the presence, location and/or amount of CD38-expressing cells in a subject comprising the steps of:
i) detecting, in a subject to whom a detectable quantity of an anti-CD38 single-domain antibody directly or indirectly coupled to a signal-emitting molecule has been administered, signal emitted by said signal-emitting molecule coupled to said antibody; and
ii) generating an image representative of the location and/or quantity or intensity of said signal.

In certain preferred embodiments, the antibody may comprise an amino acid sequence that comprises the 3 above-defined complementary determining regions CDR1 to CDR3.

These and further aspects and preferred embodiments of the invention are described in the following sections and in the appended claims. The subject-matter of the appended claims is hereby specifically incorporated in this specification.

### BRIEF DESCRIPTION OF DRAWINGS

FIG. 1 illustrates large-scale production and purification of Nb2F8, for which the genetic DNA sequence of Nb2F8 was cloned between NcoI and BstEII restriction sites in the pHEN6 vector (A), a dedicated phagemid for nanobody (Nb) expression. During this cloning step, a C-terminal hexahistidine-tag is incorporated after the Nb sequence. Cloning and subsequent quality control of the obtained pHEN6 vectors by PCR and DNA sequencing was performed. After production and purification, the Nb yield and purity were determined using SDS-PAGE followed by Coomassie blue staining and mass spectrometry analysis. A single band around 14 kDa was observed (B), corresponding to the theoretical molecular weight of nanobodies. The amino acid sequence of nanobody 2F8 is given in (C). The purified Nbs were used in flow cytometry and detected by a Phycoerythrin-labelled anti-His Ab as a secondary antibody, confirming binding to human CD38 receptor, expressed on RPMI-8226 cells (D). The anti-CD38 nanobodies recognized CD38⁺ multiple myeloma (MM) cell lines (e.g., RPMI-8226) and CD38⁺ Non-Hodgkin lymphoma cells, while no binding was seen with the CD38⁻ cell line or with an irrelevant nanobody (E). The CD38-specific Nb2F8 (conjugated to a His-tag) was used as primary antibody and an APC-labelled anti-His Ab as a secondary antibody. Cell lines: K562 human chronic myelogenous leukemia cell line, LB Non-Hodgkin lymphoma, LP1 human multiple myeloma cell line, OPM2 human multiple myeloma cell line (OPM2 cells express a very low level of CD38 compared to the other positive cell lines), RPMI-8226 human multiple myeloma cell line, U266+(CD38+ flow sorted U266) human multiple myeloma cell line. (F) Calculated K_{D} values of all 4 anti-CD38 nanobodies.

FIG. 2 illustrates flow cytometry examination of potential interference between nanobodies Nb375, Nb1053, Nb551 or Nb2F8 and daratumumab for binding on CD38. (A) Flow cytometry results (% of parent is the percentage of cells analysed among all the cells present in the tube, without dead cells and cell debris; (B, C) Biolayer Interferometry results.

FIG. 3 illustrates SPECT/CT scans of a mouse model bearing CD38⁺ RPMI-8226 tumors, using Technetium-99m labelled nanobodies Nb2F8, Nb1053 and a control nanobody.

FIG. 4 illustrates ex vivo biodistribution of ^{99m}Tc-anti-CD38 Nbs and ^{99m}Tc-NbCTRL in RPMI-8226 tumor mouse model after 1 hour post-injection and expressed as percentage of injected activity per g of organs, obtained after dissection.

FIG. 5 illustrates (A) determination of the affinity of ¹¹¹In-DTPA-Nb2F8 toward the CD38-receptor on CD38⁺ RPMI-8226 myeloma cells, (B) degree of internalisation of cell-associated ¹¹¹In-DTPA-Nb2F8 by RPMI-8226 cells, and (C) degree of internalisation of cell-associated His-tagged Nb2F8 nanobody by RPMI-8226 cells.

FIG. 6 illustrates in vivo (B, C) and ex vivo (A, D) biodistribution of Indium-111 labelled Nb2F8 (A, B, D) or control nanobody (C) in mice bearing CD38⁺ RPMI-8226 tumors.

FIG. 7 illustrates in vivo biodistribution of Lutetium-177 labelled Nb2F8 in mice bearing CD38⁺ RPMI-8226 tumors.

FIG. 8 illustrates the design of a therapeutic experiment in a mouse model bearing CD38⁺ RPMI-8226 tumors. Numbers indicate days.

FIG. 9 illustrates evolution of the tumor volumes of mice bearing CD38⁺ RPMI-8226 tumors after starting the treatment with ¹⁷⁷Lu labelled Nbs (2F8 and CTRL) or vehicle. (A) Evolution of tumor volumes from day 0 to day 42 post Nbs administration. (B) Waterfall plot illustrating the increase or decrease in tumor volume of each mouse at day 13 post Nbs administration.

FIG. 10 illustrates evolution of the tumor volumes (A) and survival (B) of mice bearing CD38⁺ RPMI-8226 tumours when treated with several different dose regimens of ¹⁷⁷Lu labelled Nbs (2F8) or vehicle.

FIG. 11 illustrates a pre-targeting strategy using Nb2F8, in which a synthetic oligonucleotide sequence (PNA1) is conjugated by Sortase A to the 2F8 nanobody after recognition of an LPETG site that was added during nanobody production (A). A second oligonucleotide sequence (PNA2) complementary to PNA1 is provided conjugated to tetraxetan (DOTA) chelating a suitable radionuclide, such as Gallium 68 (⁶⁸Ga-DOTA chelate) (B). This chelate will hybridize in vivo to form the nanobody-PNA-DOTA complex (C).

FIG. 12 shows the vector map of pHEN6 containing the Nb2F8 with the sortase-recognized motif, the flexible linker and the His tag.

### DESCRIPTION OF EMBODIMENTS

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

As corroborated by the experimental section, which illustrates certain representative embodiments of the present invention, the inventors provide advantageous applications of certain anti-CD38 single-domain antibodies (sdAb) in medical imaging, disease monitoring and theranostics in neoplastic diseases. The fact that the CD38 antigen complex with the present anti-CD38 sdAbs is only minimally if at all internalised by the cells, and is therefore expected to not induce perceptible downregulation of CD38 expression on the cell membrane, allows to more reliably detect the actual distribution of CD38+ cells in a patient, not confounded by CD38 downregulation induced by the antibody. Additionally, the present anti-CD38 sdAbs do not compete with daratumumab for binding to CD38, and as both antibodies can thus bind to CD38 concomitantly, combination therapies or combined imaging/diagnostic and therapy applications employing both antibodies can be envisaged (such as for example, detection of CD38+ cancer cells with the present anti-CD38 sdAbs followed by therapeutic targeting of the cells, if detected in an amount compelling such intervention, using daratumumab).

Accordingly, aspects of the present invention relate to an anti-CD38 single-domain antibody directly or indirectly coupled to a second molecule for use in a method of diagnosis or monitoring a neoplastic disease in a subject, or for use in a method of treating a neoplastic disease in a subject; and further to methods for diagnosis or monitoring a neoplastic disease in a subject comprising administering to the subject an anti-CD38 single-domain antibody directly or indirectly coupled to a second molecule; to an anti-CD38 single-domain antibody directly or indirectly coupled to a second molecule for use in a method of diagnosis or monitoring and treating a neoplastic disease in a subject; to methods for diagnosis or monitoring and treating a neoplastic disease in a subject comprising administering to the subject a therapeutically effective amount of anti-CD38 single-domain antibody directly or indirectly coupled to a second molecule; as well as to imaging methods for diagnosis or monitoring the presence, location and/or amount of CD38-expressing cells in a subject comprising the steps of detecting, in a subject to whom a detectable quantity of an anti-CD38 single-domain antibody directly or indirectly coupled to a signal-emitting molecule has been administered, signal emitted by said signal-emitting molecule coupled to said antibody, and generating an image representative of the location and/or quantity or intensity of said signal.

The anti-CD38 single-domain antibodies (sdAb) employed in particular embodiments of the aspects of the invention contemplated herein comprise an amino acid sequence that comprises 3 complementary determining regions (CDR1 to CDR3):

wherein CDR1 is chosen from the group consisting of:
a)

(SEQ ID NO: 1)
YTDSDYI,

b) Polypeptides that have at least 80% amino acid sequence identity with SEQ ID NO: 1,
c) Polypeptides that have 3, 2 or 1 amino acid difference with SEQ ID NO: 1,

wherein CDR2 is chosen from the group consisting of:
a)

(SEQ ID NO: 2)
TIYIGGTYIH,

b) Polypeptides that have at least 80% amino acid sequence identity with SEQ ID NO: 2,
c) Polypeptides that have 3, 2 or 1 amino acid difference with SEQ ID NO: 2,

and wherein CDR3 is chosen from the group consisting of:
a)

(SEQ ID NO: 3)
AATKWRPFISTRAAEYNY,

b) Polypeptides that have at least 80% amino acid sequence identity with SEQ ID NO: 3,
c) Polypeptides that have 3, 2 or 1 amino acid difference with SEQ ID NO: 3.

Cluster Of Differentiation 38 (CD38) molecule, or CD38 in short, also known as cyclic ADP-Ribose Hydrolase 1 or ADPRC1, is a 46-kDa type II membrane glycoprotein with a short N-terminal sequence, a single transmembrane segment and a >250-amino acid catalytic carboxyl domain. Human CD38 is annotated under U.S. government's National Center for Biotechnology Information (NCBI) Genbank (http://www.ncbi.nlm.nih.gov/) Gene ID no. 952. A human wild-type CD38 amino acid sequence may be as annotated under Genbank accession no: NP_001766.2 or Swissprot/Uniprot (http://www.uniprot.org/) accession no: P28907-i (v2), the NP_001766.2 sequence reproduced here below (the N-terminal cytoplasmic, transmembrane, and C-terminal extracellular parts of the molecule as annotated in the aforementioned database entries are shown in italics, underlined, and standard fonts, respectively):

(SEQ ID NO: 5)
MANCEFSPVSGDKPCCRLSRRAQLCLGVSILVL
ILVVVLAVVVPRWRQQWSGPGTTKRFPETVLAR
CVKYTEIHPEMRHVDCQSVWDAFKGAFISKHPC
NITEEDYQPLMKLGTQTVPCNKILLWSRIKDLA
HQFTQVQRDMFTLEDTLLGYLADDLTWCGEFNT
SKINYQSCPDWRKDCSNNPVSVFWKTVSRRFAE
AACDVVHVMLNGSRSKIFDKNSTFGSVEVHNLQ
PEKVQTLEAWVIHGGREDSRDLCQDPTIKELES
IISKRNIQFSCKNIYRPDKFLQCVKNPEDSSCT
SEI

CD38 as intended herein may particularly concern human CD38. The qualifier “human” as used herein in connection with a CD38 protein may in a certain interpretation refer to the amino acid sequence of the CD38 protein. For example, a CD38 protein having the amino acid sequence as a CD38 protein found in humans may also be obtained by technical means, e.g., by recombinant expression, cell-free translation, or non-biological peptide synthesis. Because the present sdAbs are intended to diagnostically and/or therapeutically target CD38 in humans, in a certain other interpretation the qualifier “human” may more particularly refer to a CD38 protein as found in or present in humans, regardless of whether the CD38 protein forms a part of or has been at least partly isolated from human subjects, organs, cells, or tissues. A skilled person understands that the amino acid sequence of a given native protein such as a CD38 protein may differ between or within different individuals of the same species due to normal genetic diversity (allelic variation, polymorphism) within that species and/or due to differences in post-transcriptional or post-translational modifications. Any such variants or isoforms of the native protein are subsumed by the reference to or designation of the protein.

CD38 serves as a differentiation antigen on cell surface, and also as the dominant signalling enzyme responsible for the metabolism of two intracellular calcium messenger molecules, cyclic ADP-ribose (cADPR) and nicotinic acid adenine dinucleotide phosphate (NAADP). The CD38 C-terminal domain possesses all the catalytic activities of the enzyme, and intracellular CD38 and even type III CD38 with the catalytic C-domain facing the cytosol have been reported. As a surface antigen, CD38 serves as a receptor for ligands such as CD44 and CD316.

CD38 is ubiquitously expressed in many cells, especially in the immune cells, such as lymphocytes and monocytes. CD38 expression has been found to be extremely high in some malignant cells, including hematological cancers, such as in particular multiple myeloma (MM) and chronic lymphoid leukaemia. Considering the large differences of CD38 expression between normal and myeloma cells, CD38 emerged as a suitable drug target for cancer therapy, and the anti-CD38 human monoclonal antibody, daratumumab (Darzalex™), has been approved by the EMA and US FDA for MM.

As used herein, the term “antibody” is used in the broadest sense and generally refers to an immunologic binding agent. The term encompasses whole immunoglobulin molecules, immunologically effective fragments of immunoglobulins, i.e., fragments displaying the ability to specifically bind the antigen recognised by the whole immunoglobulin molecule, as well as constructs comprising an antigen-binding portion comprised within a modified immunoglobulin-like framework, and constructs comprising an antigen-binding portion comprised within a non-immunoglobulin-like framework or scaffold. Antibody fragments comprise a portion of an intact antibody comprising the antigen-binding or variable region thereof. Examples of antibody fragments include Fab, Fab′, F(ab′)2, Fv, and single-domain sdFv (sdFv) antibodies, such as VL, VH or VHH single-domain antibodies. Fusions proteins of the heavy (VH) and light (VL) chain variable regions, commonly known as single chain Fv (scFv), are also included in antibody fragments. The term “antibody” thus includes without limitation intact monoclonal antibodies, intact polyclonal antibodies, multivalent (e.g., 2-, 3- or more-valent) antibodies and/or multi-specific (e.g., bi- or more-specific) antibodies formed from at least two intact antibodies, and further immunologically effective fragments of any of such antibodies as well as multivalent and/or multi-specific composites of such fragments (e.g., diabodies, triabodies, tetrabodies, multibodies). The term further encompasses without limitation intact antibodies and antibody fragments of non-human animal origin, as well as chimeric, humanised or chimeric/humanised forms of such antibodies or antibody fragments, and further encompasses fully human antibodies or antibody fragments. More broadly, grafting of at least one complementarity-determining region (CDR) from an antibody of one origin onto a framework of another origin is contemplated. The term “antibody” also encompasses any fusion proteins, protein conjugates or protein complexes comprising an immunoglobulin molecule or an immunologically effective fragment thereof, as well as chemically and/or enzymatically modified or derivatised immunoglobulin molecules or immunologically effective fragments thereof. The term “antibody” is not only inclusive of antibodies generated by methods comprising immunisation, but also includes any polypeptide which is made to encompass at least one CDR capable of specifically binding to an epitope on a cognate antigen, regardless whether such molecules are produced in vitro, in cell culture, or in vivo. For example, antibodies produced by recombinant DNA techniques in cultured host cells (e.g., bacterial, yeast or fungal, plant or animal cells) or in non-human host organisms (e.g., in transgenic plants or transgenic animals) are also encompassed.

The aspects disclosed herein employ an anti-CD38 single-domain antibody. The term “domain” (of a polypeptide or protein) as used herein refers to a folded protein structure which has the ability to retain its tertiary structure independently of the rest of the protein. Generally, domains are responsible for discrete functional properties of proteins, and in many cases may be added, removed or transferred to other proteins without loss of function of the remainder of the protein and/or of the domain. The term as contemplated here is particularly used to denote an “immunoglobulin domain”, a globular region of an antibody chain (such as for example a chain of a conventional 4-chain antibody or of a heavy chain antibody), or to a polypeptide that essentially consists of or consists of such a globular region. Structurally, immunoglobulin domains have been described as retaining the immunoglobulin fold characteristic of antibody molecules, which in particular involves a two-layer sandwich of about seven antiparallel beta-strands arranged in two beta-sheets, optionally stabilised by a conserved disulphide bond. Particularly intended by reference to a “domain” is the immunoglobulin variable domain. Variable domains have been described to consist essentially of four “framework regions” which are referred to in the art and herein below as “framework region 1” or “FR1”, “framework region 2” or “FR2”, “framework region 3” or “FR3”, and “framework region 4” or “FR4”, respectively; which framework regions are interrupted or interjected by three “complermentarity determining regions” or “CDRs”, which are referred to in the art and herein below as “complementarity determining region 1” or “CDR1”, “complementarity determining region 2” or “CDR2”, and “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) that confer specificity to an antibody for the antigen by carrying the antigen-binding site, with CDRs (also known as “hypervariable regions”) being the portions of the variable chain which bind to and interact with an epitope of the antigen.

The terms “single-domain” or “single variable domain” or “immunoglobulin single variable domain” defines molecules wherein the antigen-binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e., a total of 6 CDRs will be involved in antigen binding site formation. Hence, the antigen-binding domain of a conventional 4-chain antibody (such as an IgG, IgM, IgA, IgD or IgE molecule) or of a Fab fragment, a F(ab′)2 fragment, an Fv fragment such as a disulphide linked Fv or a scFv fragment, or a diabody derived from such conventional 4-chain antibody, would normally not be regarded as an immunoglobulin single variable domain, since in these cases binding to the respective epitope of an antigen would normally not occur by one (single) immunoglobulin domain but by a pair of (associated) immunoglobulin domains such as light and heavy chain variable domains, i.e., by a VH-VL pair of immunoglobulin domains, which jointly bind to an epitope of the respective antigen.

In contrast, immunoglobulin single variable domains are capable of specifically binding to an epitope of the antigen without pairing with an additional immunoglobulin variable domain. The binding site of an immunoglobulin single variable domain is formed by a single VH, VHH or VL domain. Hence, the antigen binding site of an inununoglobulin single variable domain is formed by no more than three CDRs. An immunoglobulin single variable domain may be a light chain variable domain (a VL-sequence) or a suitable fragment thereof, or a heavy chain variable domain (a VH-sequence or a VHH-sequence) or a suitable fragment thereof, insofar capable of forming a single antigen binding unit (i.e., a functional antigen binding unit that essentially consists of the single variable domain, such that the single antigen binding domain does not need to interact with another variable domain to form a functional antigen binding unit).

Immunoglobulin single variable domains in their broadest sense are not limited to a specific biological source or to a specific method of preparation. The term “immunoglobulin single variable domain” encompasses variable domains of different origin, comprising mouse, rat, rabbit, donkey, human, shark (for example, the so-called “IgNAR domains”, see for example WO 05/18629), and camelid variable domains.

Hence, as contemplated herein, the operative, antigen-binding principle of a single-domain antibody is an immunoglobulin single variable domain. In certain preferred embodiments, this single-domain may be a “heavy chain variable domain” which, as used herein, denotes (i) the variable domain derived from the heavy chain of a heavy-chain antibody, which is naturally devoid of light chains, including but not limited to the variable domain of the heavy chain of heavy-chain antibodies of camelids or sharks or (ii) the variable domain derived from the heavy chain of a conventional four-chain antibody (also indicated hereafter as V_{H}), including but not limited to a camelised (as further defined herein) variable domain of the heavy chain of a conventional four-chain antibody (also indicated hereafter as camelised V_{H}), or any functional fragments thereof. In certain preferred embodiments, the single-domain may be as stated in (i). Hence, in certain embodiments, the single-domain antibody as intended herein is a heavy chain variable domain derived from a heavy-chain antibody (V_{HH}) or a functional fragment thereof, i.e., a CD38-binding fragment thereof. Such fragment may for example bind CD38 with K_{D} not higher than 10× the K_{D} of the full-length reference, preferably not higher than 5× the K_{D} of the full-length reference, more preferably not higher than 2× the K_{D} of the full-length reference, such as with K_{D} substantially the same as the K_{D} of the full-length reference (e.g., +/−1.5× or +/−1.2×). “VHH domains”, also known as VHHs, VHH domains, VHH antibody fragments, and VHH antibodies, have originally been described as the antigen binding immunoglobulin (variable) domain of “heavy-chain antibodies”, i.e., of antibodies devoid of light chains (Hamers-Casterman et al. Naturally occurring antibodies devoid of light chains. Nature 1993, vol. 363, 446-448). The term “VHH domain” has been chosen in order to distinguish these variable domains from the heavy chain variable domains that are present in conventional 4-chain antibodies (which are referred to conventionally and herein as “VH domains”) and from the light chain variable domains that are present in conventional 4-chain antibodies (which are referred to conventionally and herein as “VL domains”). Hence, in certain preferred embodiments, the single-domain may be a VHH domain, or in other words the anti-CD38 antibody may be a VHH single-domain antibody, or VHH antibody in short.

In certain preferred embodiments, the anti-CD38 antibody may be a nanobody. The terms “nanobody” (Nb) as used herein (“Nanobody®”, “Nanobodies®” and “Nanoclone®” are registered trademarks of Ablynx N.V., Ghent, Belgium) refers to a single variable domain derived from naturally occurring heavy-chain antibodies (devoid of light chains), in particular those found in camelids (Hamers-Casterman et al. 1993, supra; Desmyter et al. Crystal structure of a camel single-domain VH antibody fragment in complex with lysozyme. Nat Struct Biol. 1996, vol. 3, 803-811), and consequently often referred to as VHH antibody or VHH. Camelids comprise old world camelids (Camelus bactrianus and Camelus dromedarius) and new world camelids (for example Lama paccos, Lama glama, Lama guanicoe and Lama vicugna). The term nanobody as used herein in its broadest sense is not limited to a specific biological source or to a specific method of preparation. For example, nanobodies in the broadest sense may encompass an immunological binding agent obtained: (1) by isolating the VHH domain of a naturally occurring heavy-chain antibody; (2) by expression of a nucleotide sequence encoding a naturally occurring VHH domain; (3) by “humanisation” of a naturally occurring VHH domain or by expression of a nucleic acid encoding a such humanised VHH domain; (4) by “camelisation” of a naturally occurring VH domain from any animal species, and in particular from a mammalian species, such as from a human being, or by expression of a nucleic acid encoding such a camelised VH domain; (5) by “camelisation” of a “domain antibody” or “Dab” as described in the art or by expression of a nucleic acid encoding such a camelised VH domain; (6) by using synthetic or semi-synthetic techniques for preparing proteins, polypeptides or other amino acid sequences known per se; (7) by preparing a nucleic acid encoding a nanobody using techniques for nucleic acid synthesis known per se, followed by expression of the nucleic acid thus obtained; and/or (8) by any combination of one or more of the foregoing.

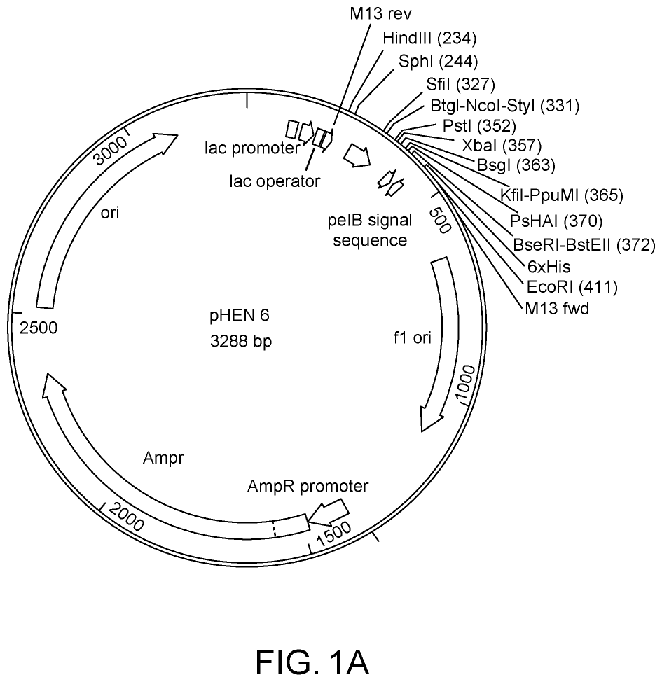

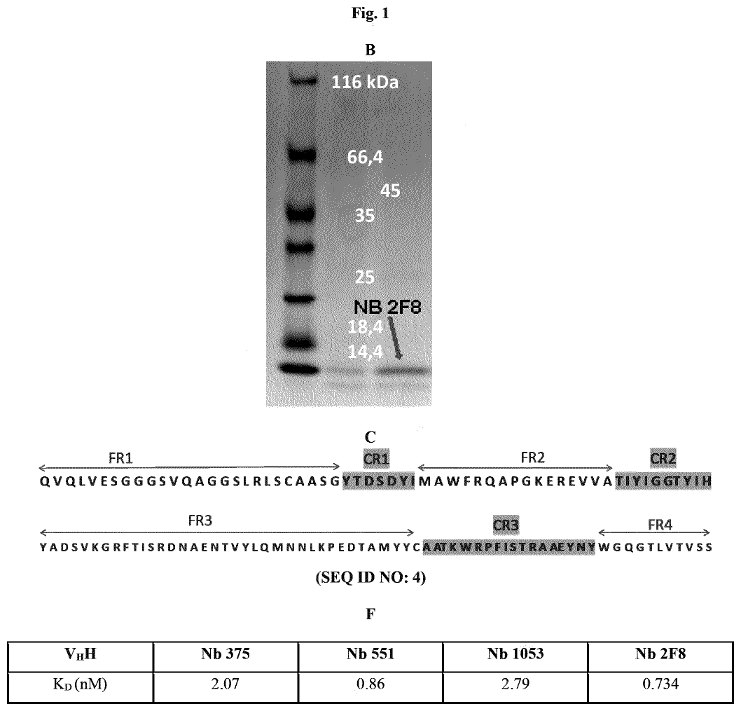

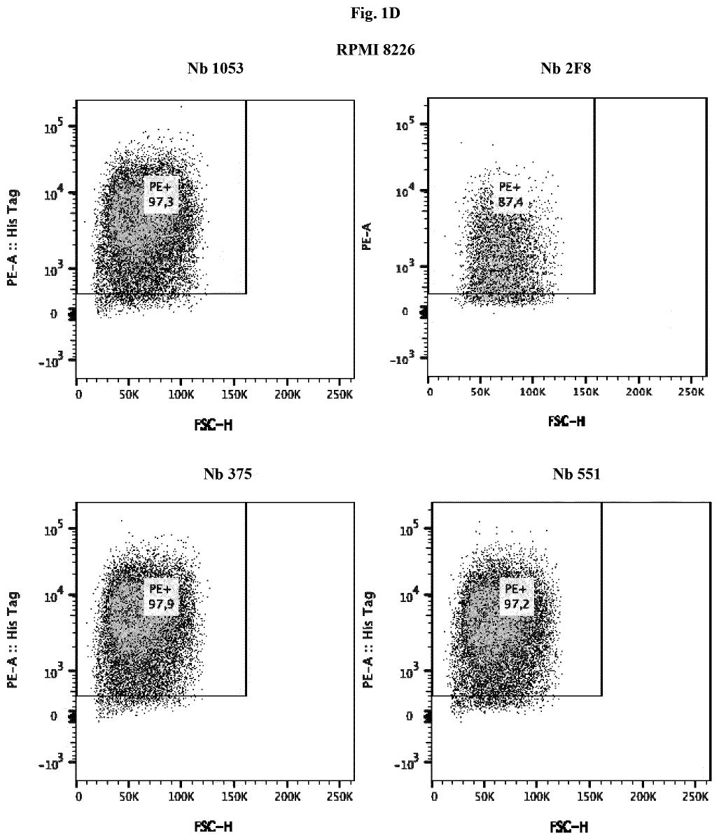

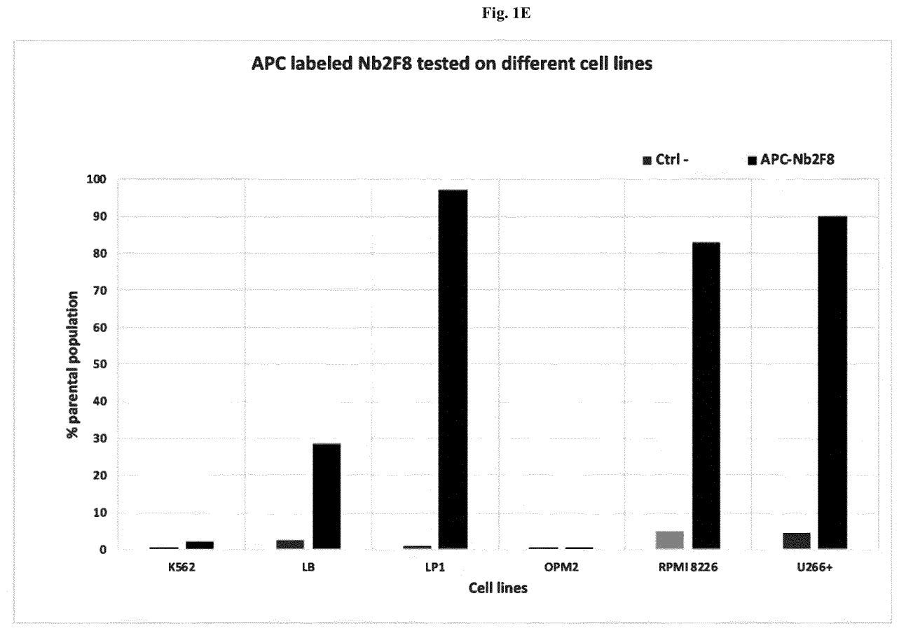

As explained above for variable domains in general, the amino acid sequence and structure of a nanobody can be considered—without however being limited thereto—to be comprised of four framework regions, FR1 to FR4, interjected by three complementarity determining regions, CDR1 to CDR3 (see for example FIG. 1C). The total number of amino acid residues in a nanobody can be in the region of 110-130, preferably 110-120, such as preferably 112-115. However, parts, fragments, analogues or derivatives of a nanobody are not particularly limited as to their length and/or size, as long as such parts, fragments, analogues or derivatives meet the further requirements outlined herein and are preferably suitable for the purposes described herein.

For a further description of VHHs and nanobodies, reference is made to the review article by Muyldermans (Reviews in Molecular Biotechnology 74: 277-302, 2001), as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1433793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V.

In certain preferred embodiments, the anti-CD38 antibody may be a domain antibody (Dab). “Domain antibodies”, also known as “Dabs” or “dAbs” (“Domain Antibody®”, “Domain Antibodies®”, “dAb®” and “dAbs®” are registered trademarks of the GlaxoSmithKline group of companies) have been described for example in EP 0368684, Ward et al. (Nature 1989, vol. 341, 544-546), Holt et al. (Tends in Biotechnology 2003, vol. 21, 484-490) and WO 03/002609 as well as for example WO 04/068820, WO 06/030220, WO 06/003388 and other published patent applications of Domantis Ltd. Domain antibodies essentially correspond to the VH or VL domains of non-camelid mammals, in particular human 4-chain antibodies. In order to bind an epitope as a single antigen binding domain, i.e., without being paired with a VL or VH domain, respectively, specific selection for such antigen binding properties is required, e.g., by using libraries of human single VH or VL domain sequences. Domain antibodies have, like VHHs, a molecular weight of approximately 13 to approximately 16 kDa and, if derived from fully human sequences, do not require humanisation, e.g., for administration to humans.

Immunoglobulin single variable domains such as domain antibodies and nanobodies (including VHH domains) can be subjected to humanisation. In particular, humanised immunoglobulin single variable domains, such as nanobodies (including VHH domains) may be immunoglobulin single variable domains that are as generally defined in the previous paragraphs, but in which at least one amino acid residue is present (and in particular, at least one framework residue) that is and/or that corresponds to a humanising substitution. Potentially useful humanising substitutions can be ascertained by comparing the sequence of the framework regions of a naturally occurring VHH sequence with the corresponding framework sequence of one or more closely related human VH sequences, after which one or more of the potentially useful humanising substitutions (or combinations thereof) thus determined can be introduced into said VHH sequence (in any manner known per se) and the resulting humanised VHH sequences can be tested for affinity for the target, for stability, for ease and level of expression, and/or for other desired properties. In this way, by means of a limited degree of trial and error, other suitable humanising substitutions (or suitable combinations thereof) can be determined by the skilled person based on the disclosure herein. Also, based on the foregoing, (the framework regions of) an immunoglobulin single variable domain, such as a nanobody (including VHH domains) may be partially humanised or fully humanised. Hence, in certain preferred embodiments, the anti-CD38 single-domain antibody may be a humanised VHH antibody. In certain embodiments, anti-CD38 single-domain antibody may be a human or humanised domain antibody.

Immunoglobulin single variable domains such as domain antibodies and nanobodies (including VHH domains and humanised VHH domains), can also be subjected to affinity maturation by introducing one or more alterations in the amino acid sequence of one or more CDRs, which alterations result in an improved affinity of the resulting immunoglobulin single variable domain for its respective antigen, as compared to the respective parent molecule. Affinity-matured immunoglobulin single variable domain molecules may be prepared by methods known in the art, for example, as described by Marks et al. (Biotechnology 1992, vol. 10, 779-783), Barbas et al. (Proc. Nat. Acad. Sci USA 1994, vol. 91, 3809-3813), Shier et al. (Gene 1995, vol. 169, 147-155), Yelton et al. (Immunol. 1995, vol. 155, 1994-2004), Jackson et al. (J. Immunol. 1995, vol. 154, 3310-9), Hawkins et al. (J. Mol. Biol. 1992, vol. 226, 889 896, 1992), and Johnson and Hawkins (Affinity maturation of antibodies using phage display, Oxford University Press, 1996).

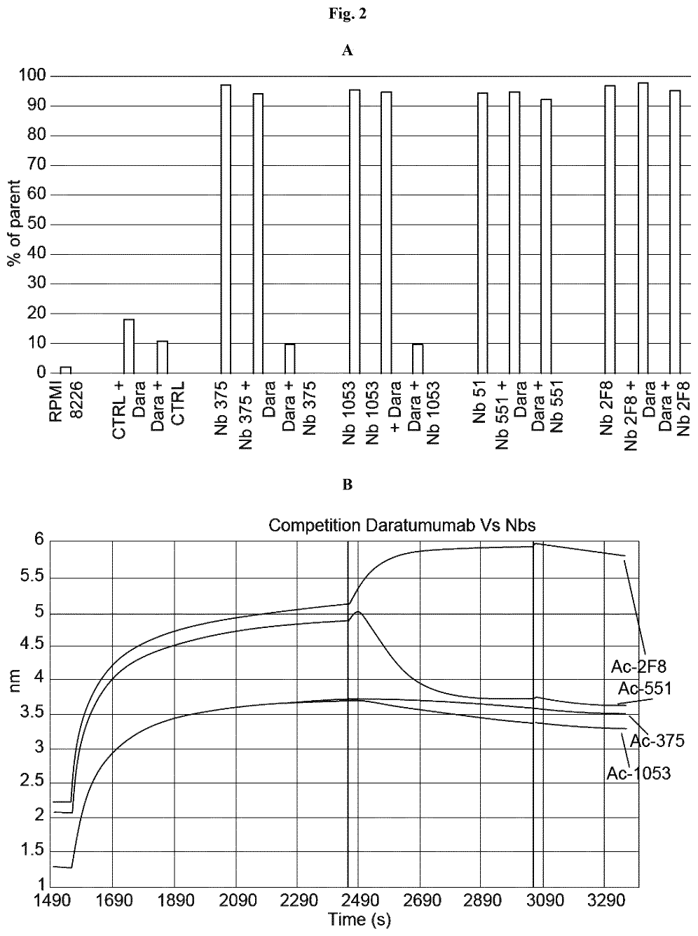

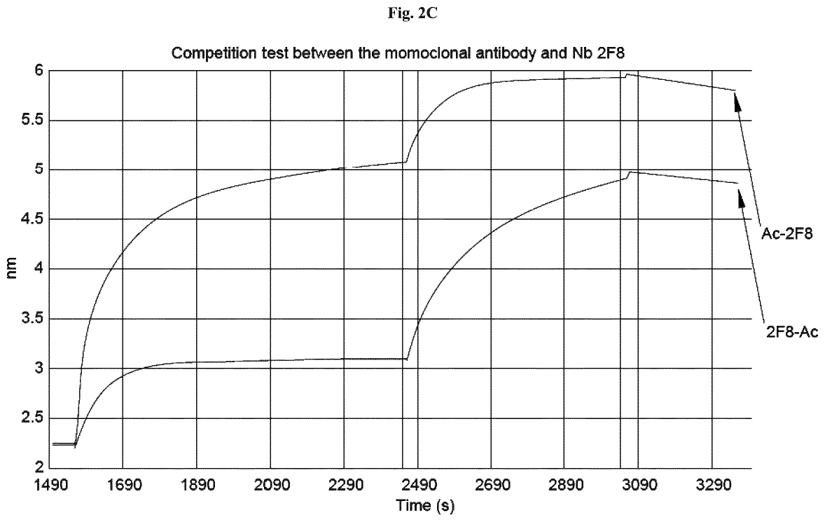

To convey the position of CDRs and FRs in a single-domain antibody such as a VHH, the amino acid residues of the antibody may be numbered according to a suitable numbering system. For example, a general numbering system for VH domains was formulated by Kabat et al. (“Sequence of proteins of immunological interest”, US Public Health Services, NIH Bethesda, Md., Publication No. 91), and has been applied to VHH domains from camelids, as shown, e.g., in FIG. 2 of Riechmann and Muyldermans (Single domain antibodies: comparison of camel VH and camelised human VH domains. J. Immunol. Methods 1999, vol. 231, 25-38). According to this numbering, FR1 of a VHH domain comprises the amino acid residues at positions 1-30, CDR1 of a VHH domain comprises the amino acid residues at positions 31-35, FR2 of a VHH domain comprises the amino acids at positions 36-49, CDR2 of a VHH domain comprises the amino acid residues at positions 50-65, FR3 of a VHH domain comprises the amino acid residues at positions 66-94, CDR3 of a VHH domain comprises the amino acid residues at positions 95-102, and FR4 of a VHH domain comprises the amino acid residues at positions 103-113. It should be noted that as is well known in the art for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. Nonetheless, it can be said that, according to the numbering of Kabat and irrespective of the number of amino acid residues in the CDRs, position 1 according to the Kabat numbering corresponds to the start of FR1 and vice versa, position 36 according to the Kabat numbering corresponds to the start of FR2 and vice versa, position 66 according to the Kabat numbering corresponds to the start of FR3 and vice versa, and position 103 according to the Kabat numbering corresponds to the start of FR4 and vice versa.

The reference to single-domain antibodies including VHH and domain antibodies also encompasses functional fragments thereof that retain at least part of or the entirety of the functional activity and/or retain at least part of or the entirety of the binding specificity of the original immunoglobulin single variable domain such as VHH domain from which the fragments are derived. Functional fragments are not particularly limited as to their length and/or size, and may without limitation denote N-terminally and/or C-terminally deleted or truncated forms of the original immunoglobulin single variable domain which may for example represent at least about 50% (by amino acid number), e.g., at least about 60%, or at least about 70%, or at least about 80%, or at least about 90%, or at least about 910%, or at least about 92%, or at least about 93%, or at least about 94%, or at least about 95%, or at least about 96%, or at least about 97%, or at least about 98%, or at least about 99% of the contiguous amino acid sequence of said original immunoglobulin single variable domain. The term encompasses fragments arising by any mechanism, such as, without limitation, by heterologous expression of a truncated form of the immunoglobulin single variable domain, or by physical, chemical or enzymatic proteolysis thereof. Usually, a functional fragment of an immunoglobulin single variable domain such as a VHH domain as disclosed herein contains at least some of the amino acid residues that form at least one of the complementarity determining regions of the original immunoglobulin single variable domain such as VHH domain from which they are derived from.

The antibodies contemplated herein are anti-CD38 antibodies, i.e., the antibodies specifically bind to CD38. The term “specifically bind” as used throughout this specification means that an agent binds to one or more desired molecules or analytes substantially to the exclusion of other molecules which are random or unrelated, and optionally substantially to the exclusion of other molecules that are structurally related. Put differently, an antibody is said to specifically bind an antigen when it preferentially recognises its target antigen in a complex mixture of proteins and/or macromolecules.

The binding of an antibody would in particular be to an epitope on the CD38 protein. The term “epitope” includes any polypeptide determinant capable of specifically binding to an immunoglobulin or T-cell receptor. Epitope determinants may include chemically active surface groupings of molecules such as amino acids, sugar side chains, phosphoryl, or sulfonyl, and may have specific three dimensional structural characteristics, and/or specific charge characteristics. An epitope is a region of an antigen that is bound by an antibody. An antibody is said to specifically bind an antigen when it preferentially recognises its target antigen in a complex mixture of proteins and/or macromolecules. In certain preferred embodiments, the antibody's epitope may be on a portion of CD38 exposed on the cells surface, such as in particular the epitope may be comprised by the C-terminal extracellular part of CD38.

The term “specificity” refers to the number of different types of antigens or antigenic determinants to which a particular antigen-binding molecule or antigen-binding protein (such as an antibody) molecule can bind. The specificity of an antigen-binding protein can be determined based on affinity and/or avidity. The affinity, represented by the equilibrium constant for the dissociation of an antigen with an antigen-binding protein (KD), is a measure for the binding strength between an antigenic determinant and an antigen-binding site on the antigen-binding protein: the lesser the value of the KD, the stronger the binding strength between an antigenic determinant and the antigen-binding molecule (alternatively, the affinity can also be expressed as the affinity constant (KA), which is 1/KD). As will be clear to the skilled person, affinity can be determined in a manner known per se, depending on the specific antigen of interest. Avidity is the measure of the strength of binding between an antigen-binding molecule (such as an antibody) and the pertinent antigen. Avidity is related to both the affinity between an antigenic determinant and its antigen binding site on the antigen-binding molecule and the number of pertinent binding sites present on the antigen-binding molecule. Typically, antigen-binding proteins (such as antibodies) will bind with a dissociation constant (KD) of 1×10⁻⁵ to 1×10⁻¹² moles/liter (M) or less, and preferably 1×10⁻⁷ to 1×10⁻¹² M or less, and more preferably 1×10⁻⁸ to 1×10⁻¹² M or less and even more preferably 1×10⁻⁹ to 1×10⁻¹² M or less, such as between 1×10⁻⁹ and 1×10⁻¹⁰ M, or between 1×10⁻¹⁰ and 1×10⁻¹⁰ M, wherein KD=[AB][AG]/[AB-AG], AB denotes the antibody, AG denotes the antigen, and AB-AG denotes the antibody-antigen complex. Any Kl value greater than 10⁻⁴ M is generally considered to indicate non-specific binding. Preferably, an antibody will bind to the desired antigen with an KD less than 500 nM, preferably less than 200 nM, more preferably less than 10 nM, such as less than 5 nM, less than 4 nM, less than 3 nM, less than 2 nM, or less than 1 nM, e.g., about 500 pM, about 600 pM, about 700 pM, about 800 pM, or about 900 pM. In certain preferred examples, an antibody will bind to the desired antigen with an KD of between 500 pM and 3 nM. Specific binding of an antigen-binding protein to an antigen or antigenic determinant can be determined in any suitable manner known per se, including, for example, Scatchard plot analysis and/or competitive binding assays, such as radioimmunoassays (RIA), enzyme immunoassays (EIA) and sandwich competition assays, and the different variants thereof known per se in the art.

The anti-CD38 single-domain antibodies employed in particular embodiments of the aspects of the invention contemplated herein are defined by the sequences of the CDRs (CDR1, CDR2, CDR3) comprised by their respective amino acid sequences. The sequence of CDR1 is YTDSDYI (SEQ ID NO: 1), or the sequence of CDR1 displays at least 80% amino acid sequence identity with SEQ ID NO: 1, or the sequence of CDR1 displays 3, 2 or 1 amino acid difference with SEQ ID NO: 1. Preferably, the sequence of CDR1 displays at least 85%, more preferably at least 90%, even more preferably at least 95% sequence identity with SEQ ID NO: 1. Preferably, the sequence of CDR1 displays at most 2, more preferably at most 1 amino acid difference with SEQ ID NO: 1. Most preferably the sequence of CDR1 is as set forth in SEQ ID NO: 1. The sequence of CDR2 is TIYIGGTYIH (SEQ ID NO: 2), or the sequence of CDR2 displays at least 80% amino acid sequence identity with SEQ ID NO: 2, or the sequence of CDR2 displays 3, 2 or 1 amino acid difference with SEQ ID NO: 2. Preferably, the sequence of CDR2 displays at least 85%, more preferably at least 90%, even more preferably at least 95% sequence identity with SEQ ID NO: 2. Preferably, the sequence of CDR2 displays at most 2, more preferably at most 1 amino acid difference with SEQ ID NO: 2. Most preferably the sequence of CDR2 is as set forth in SEQ ID NO: 2. The sequence of CDR3 is AATKWRPFISTRAAEYNY (SEQ ID NO: 3), or the sequence of CDR3 displays at least 80% amino acid sequence identity with SEQ ID NO: 3, or the sequence of CDR3 displays 3, 2 or 1 amino acid difference with SEQ ID NO: 3. Preferably, the sequence of CDR3 displays at least 85%, more preferably at least 90%, even more preferably at least 95% sequence identity with SEQ ID NO: 3. Preferably, the sequence of CDR3 displays at most 2, more preferably at most 1 amino acid difference with SEQ ID NO: 3. Most preferably the sequence of CDR3 is as set forth in SEQ ID NO: 3.

Accordingly, in certain particularly preferred embodiments, the amino acid sequence of CDR1 of the anti-CD38 single-domain antibody is YTDSDYI (SEQ ID NO: 1), the amino acid sequence of CDR2 is TIYIGGTYIH (SEQ ID NO: 2), and the amino acid sequence of CDR3 is

(SEQ ID NO: 3)
AATKWRPFISTRAAEYNY.

In certain particularly preferred embodiments, the anti-CD38 single-domain antibody may comprise, consist essentially of or consists of an amino acid sequence having a sequence identity of at least 80% to SEQ ID NO: 4 or a functional fragment thereof:

(SEQ ID NO: 4)
QVQLVESGGGSVQAGGSLRLSCAASGYTDSDYI
MAWFRQAPGKEREVVATIYIGGTYIHYADSVKG
RFTISRDNAENTVYLQMNNLKPEDTAMYYCAAT
KWRPFISTRAAEYNYWGQGTLVTVSS.

SEQ ID NO: 4 comprises CDR1, CDR2 and CDR3 as set forth in SEQ ID NO: 1, 2 and 3, indicated above in underlined, bold, and underlined bold fonts, respectively. In certain embodiments, the anti-CD38 single-domain antibody may comprise, consist essentially of or consists of an amino acid sequence having a sequence identity of at least 810%, at least 82%, at least 83% or at least 84% to SEQ ID NO: 4, preferably a sequence identity of at least 85%, such as at least 86%, at least 87%, at least 88% or at least 89% to SEQ ID NO: 4, more preferably a sequence identity of at least 90%, such as at least 91%, at least 92%, at least 93% or at least 94% to SEQ ID NO: 4, even more preferably a sequence identity of at least 95%, such as at least 96%, at least 97%, at least 98% or at least 99% to SEQ ID NO: 4, or a functional fragment thereof.

The framework regions comprised by the antibody as set forth in SEQ ID NO: 4 are indicated above in italic font:

FR1:
(SEQ ID NO: 6)
QVQLVESGGGSVQAGGSLRLSCAASG,
FR2:
(SEQ ID NO: 7)
MAWFRQAPGKEREVVA,
FR3:
(SEQ ID NO: 8)
YADSVKGRFTISRDNAENTVYLQMNNLKPEDTAMYYC,
and
FR4:
(SEQ ID NO: 9)
WGQGTLVTVSS.

In certain examples, FR1, FR2, FR3 and FR4 may each independently display a sequence identity of at least 80%, such as at least 81%, at least 82%, at least 83% or at least 84%, preferably at least 85%, such as at least 86%, at least 87%, at least 88% or at least 89%, more preferably at least 90%, such as at least 91%, at least 92%, at least 93% or at least 94%, even more preferably at least 95%, such as at least 96%, at least 97%, at least 98% or at least 99% to SEQ ID NO: 6, 7, 8, and 9, respectively.

In certain embodiments, the degree of sequence variation in at least one framework region, more preferably in two or three framework regions, and more preferably in all four framework regions may be comparatively higher than the degree of sequence variation in at least one CDR, preferably in two CDRs, and more preferably in all three CDRs. Hence, in certain embodiments, the degree of sequence variation in the framework regions may be comparatively higher than the degree of sequence variation in the CDRs. By virtue of non-limiting examples:
FR1, FR2, FR3 and FR4 may each independently display a sequence identity of at least 80% to SEQ ID NO: 6, 7, 8, and 9, respectively, and CDR1, CDR2 and CDR3 may each independently display a sequence identity of at least 81%, at least 82%, at least 83% or at least 84%, preferably at least 85%, such as at least 86%, at least 87%, at least 88% or at least 89%, more preferably at least 90%, such as at least 91%, at least 92%, at least 93% or at least 94%, even more preferably at least 95%, such as at least 96%, at least 97%, at least 98% or at least 99% to SEQ ID NO: 1, 2 and 3, respectively; or
FR1, FR2, FR3 and FR4 may each independently display a sequence identity of at least 80% and preferably at least 85% to SEQ ID NO: 6, 7, 8, and 9, respectively, and CDR1, CDR2 and CDR3 may each independently display a sequence identity of at least 86%, at least 87%, at least 88% or at least 89%, preferably at least 90%, such as at least 91%, at least 92%, at least 93% or at least 94%, more preferably at least 95%, such as at least 96%, at least 97%, at least 98% or at least 99% to SEQ ID NO: 1, 2 and 3, respectively; or
FR1, FR2, FR3 and FR4 may each independently display a sequence identity of at least 80%, preferably at least 85%, and more preferably at least 90% to SEQ ID NO: 6, 7, 8, and 9, respectively, and CDR1, CDR2 and CDR3 may each independently display a sequence identity of at least 91%, at least 92%, at least 93% or at least 94%, preferably at least 95%, such as at least 96%, at least 97%, at least 98% or at least 99% to SEQ ID NO: 1, 2 and 3, respectively; or
FR1, FR2, FR3 and FR4 may each independently display a sequence identity of at least 80%, preferably at least 85%, more preferably at least 90%, and even more preferably at least 95% to SEQ ID NO: 6, 7, 8, and 9, respectively, and CDR1, CDR2 and CDR3 may each independently display a sequence identity of at least 96%, at least 97%, at least 98% or at least 99% to SEQ ID NO: 1, 2 and 3, respectively.

The term “protein” generally encompasses macromolecules comprising one or more polypeptide chains. The term “polypeptide” generally encompasses linear polymeric chains of amino acid residues linked by peptide bonds. A “peptide bond”, “peptide link” or “amide bond” is a covalent bond formed between two amino acids when the carboxyl group of one amino acid reacts with the amino group of the other amino acid, thereby releasing a molecule of water. Especially when a protein is only composed of a single polypeptide chain, the terms “protein” and “polypeptide” may be used interchangeably to denote such a protein. The terms are not limited to any minimum length of the polypeptide chain. Polypeptide chains consisting essentially of or consisting of 50 or less (≤50) amino acids, such as ≤45, ≤40, ≤35, ≤30, ≤25, ≤20, ≤15, ≤10 or ≤5 amino acids may be commonly denoted as a “peptide”. In the context of proteins, polypeptides or peptides, a “sequence” is the order of amino acids in the chain in an amino to carboxyl terminal direction in which residues that neighbour each other in the sequence are contiguous in the primary structure of the protein, polypeptide or peptide. The terms may encompass naturally, recombinantly, semi-synthetically or synthetically produced proteins, polypeptides or peptides. Hence, for example, a protein, polypeptide or peptide can be present in or isolated from nature, e.g., produced or expressed natively or endogenously by a cell or tissue and optionally isolated therefrom; or a protein, polypeptide or peptide can be recombinant, i.e., produced by recombinant DNA technology, and/or can be, partly or entirely, chemically or biochemically synthesised. Without limitation, a protein, polypeptide or peptide can be produced recombinantly by a suitable host or host cell expression system and optionally isolated therefrom (e.g., a suitable bacterial, yeast, fungal, plant or animal host or host cell expression system), or produced recombinantly by cell-free translation or cell-free transcription and translation, or non-biological peptide, polypeptide or protein synthesis. The terms also encompasses proteins, polypeptides or peptides that carry one or more co- or post-expression-type modifications of the polypeptide chain(s), such as, without limitation, glycosylation, lipidation, acetylation, amidation, phosphorylation, sulphonation, methylation, pegylation (covalent attachment of polyethylene glycol typically to the N-terminus or to the side-chain of one or more Lys residues), ubiquitination, sumoylation, cysteinylation, glutathionylation, oxidation of methionine to methionine sulphoxide or methionine sulphone, signal peptide removal, N-terminal Met removal, conversion of pro-enzymes or pre-hormones into active forms, etc. Such co- or post-expression-type modifications may be introduced in vivo by a host cell expressing the proteins, polypeptides or peptides (co- or post-translational protein modification machinery may be native to the host cell and/or the host cell may be genetically engineered to comprise one or more (additional) co- or post-translational protein modification functionalities), or may be introduced in vitro by chemical (e.g., pegylation) and/or biochemical (e.g., enzymatic) modification of the isolated proteins, polypeptides or peptides.

The term “amino acid” encompasses naturally occurring amino acids, naturally encoded amino acids, non-naturally encoded amino acids, non-naturally occurring amino acids, amino acid analogues and amino acid mimetics that function in a manner similar to the naturally occurring amino acids, all in their D- and L-stereoisomers, provided their structure allows such stereoisomeric forms. Amino acids are referred to herein by either their name, their commonly known three letter symbols or by the one-letter symbols recommended by the IUPAC-IUB Biochemical Nomenclature Commission. A “naturally encoded amino acid” refers to an amino acid that is one of the 20 common amino acids or pyrrolysine, pyrroline-carboxy-lysine or selenocysteine. The 20 common amino acids are: Alanine (A or Ala), Cysteine (C or Cys), Aspartic acid (D or Asp), Glutamic acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). A “non-naturally encoded amino acid” refers to an amino acid that is not one of the 20 common amino acids or pyrrolysine, pyrroline-carboxy-lysine or selenocysteine. The term includes without limitation amino acids that occur by a modification (such as a post-translational modification) of a naturally encoded amino acid, but are not themselves naturally incorporated into a growing polypeptide chain by the translation complex, as exemplified without limitation by N-acetylglucosaminyl-L-serine, N-acetylglucosam inyl-L-threonine, and O-phosphotyrosine. Further examples of non-naturally encoded, un-natural or modified amino acids include 2-Aminoadipic acid, 3-Aminoadipic acid, beta-Alanine, beta-Aminopropionic acid, 2-Aminobutyric acid, 4-Aminobutyric acid, piperidinic acid, 6-Aminocaproic acid, 2-Aminoheptanoic acid, 2-Aminoisobutyric acid, 3-Aminoisobutyric acid, 2-Aminopimelic acid, 2,4 Diaminobutyric acid, Desmosine, 2,2′-Diaminopimelic acid, 2,3-Diaminopropionic acid, N-Ethylglycine, N-Ethylasparagine, homoserine, homocysteine, Hydroxylysine, allo-Hydroxylysine, 3-Hydroxyproline, 4-Hydroxyproline, Isodesmosine, allo-Isoleucine, N-Methylglycine, N-Methylisoleucine, 6-N-Methyllysine, N-Methylvaline, Norvaline, Norleucine, or Ornithine. Also included are amino acid analogues, in which one or more individual atoms have been replaced either with a different atom, an isotope of the same atom, or with a different functional group. Also included are un-natural amino acids and amino acid analogues described in Ellman et al. Methods Enzymol. 1991, vol. 202, 301-36. The incorporation of non-natural amino acids into proteins, polypeptides or peptides may be advantageous in a number of different ways. For example, D-amino acid-containing proteins, polypeptides or peptides exhibit increased stability in vitro or in vivo compared to L-amino acid-containing counterparts. More specifically, D-amino acid-containing proteins, polypeptides or peptides may be more resistant to endogenous peptidases and proteases, thereby providing improved bioavailability of the molecule and prolonged lifetimes in vivo.

The term “sequence identity” with regard to amino acid sequences denotes the extent of overall sequence identity (i.e., including the whole or entire amino acid sequences as recited in the comparison) expressed in % between the amino acid sequences read from N-terminus to C-terminus. Sequence identity may be determined using suitable algorithms for performing sequence alignments and determination of sequence identity as know per se. Exemplary but non-limiting algorithms include those based on the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250), for example using the published default settings or other suitable settings (such as, e.g., for the BLASTN algorithm: cost to open a gap=5, cost to extend a gap=2, penalty for a mismatch=−2, reward for a match=1, gap x_dropoff=50, expectation value=10.0, word size=28; or for the BLASTP algorithm: matrix=Blosum62 (Henikoff et al., 1992, Proc. Natl. Acad. Sci., 89:10915-10919), cost to open a gap=11, cost to extend a gap=1, expectation value=10.0, word size=3).

An example procedure to determine the percent identity between a particular amino acid sequence and a query amino acid sequence (e.g., the sequence of an anti-CD38 antibody, or of a CDR, or of an FR) will entail aligning the two amino acid sequences each read from N-terminus to C-terminus using the Blast 2 sequences (B12seq) algorithm, available as a web application or as a standalone executable programme (BLAST version 2.2.31+) at the NCBI web site (www.ncbi.nlm.nih.gov), using suitable algorithm parameters. An example of suitable algorithm parameters includes: matrix=Blosum62, cost to open a gap=11, cost to extend a gap=1, expectation value=10.0, word size=3). If the two compared sequences share identity, then the output will present those regions of identity as aligned sequences. If the two compared sequences do not share identity, then the output will not present aligned sequences. Once aligned, the number of matches will be determined by counting the number of positions where an identical amino acid residue is presented in both sequences. The percent identity is determined by dividing the number of matches by the length of the query sequence, followed by multiplying the resulting value by 100. The percent identity value may, but need not, be rounded to the nearest tenth. For example, 78.11, 78.12, 78.13, and 78.14 may be rounded down to 78.1, while 78.15, 78.16, 78.17, 78.18, and 78.19 may be rounded up to 78.2. It is further noted that the detailed view for each segment of alignment as outputted by B12seq already conveniently includes the percentage of identities.

Where an amino acid sequence differs, varies or diverges from a certain other amino acid sequence—for example, where the former amino acid sequence is said to display a certain degree or percentage of sequence identity to the latter amino acid sequence, or where the former amino acid sequence is said to differ by a certain number of amino acids from the latter amino acid sequence—such sequence variation may be constituted by one or more amino acid additions (e.g., a single amino acid or a stretch of two or more contiguous amino acids may be added at one position of an amino acid sequence or each independently at two or more positions of an amino acid sequence), deletions (e.g., a single amino acid or a stretch of two or more contiguous amino acids may be deleted at one position of an amino acid sequence or each independently at two or more positions of an amino acid sequence), and/or or substitutions (e.g., a single amino acid or a stretch of two or more contiguous amino acids may substitute a single one or a stretch of two or more contiguous amino acids at one position of an amino acid sequence or each independently at two or more positions of an amino acid sequence).

Preferably, the one or more amino acid substitutions, in particular one or more single amino acid substitutions, may be conservative amino acid substitutions. A conservative amino acid substitution is a substitution of one amino acid for another with similar characteristics. Conservative amino acid substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (i.e., basic) amino acids include arginine, lysine and histidine. The negatively charged (i.e., acidic) amino acids include aspartic acid and glutamic acid. Any substitution of one member of the above-mentioned polar, basic, or acidic groups by another member of the same group can be deemed a conservative substitution. By contrast, a non-conservative substitution is a substitution of one amino acid for another with dissimilar characteristics.

The anti-CD38 single-domain antibody is coupled, for example directly or indirectly coupled, to a second molecule, which unlocks the medical imaging, diagnostic and/or theranostic potential and usefulness of the antibody. The reference to a second molecule is thus to be construed broadly as pertaining to any molecular entity, such as a chemical or biochemical entity, such as for example an atom, molecule, macromolecule, ion, radical or complex, that is coupled to the antibody, wherein said second molecule is detectable, in particular can be detected or visualised in the body of the subject so as to convey information on the bio-distribution of the antibody. The second molecule can also facilitate therapy of a neoplastic disease, in particular it can have a cytotoxic effect on CD38-expressing neoplastic cells bound by the antibody, reducing their viability, proliferation or destroying the cells.

The anti-CD38 sdAb and the second molecular entity may be coupled, connected or joined through chemical interactions or chemical bond or bonds between them. Whereas non-covalent interactions, such as ionic interactions, hydrogen bonds, Van der Waals interactions, chelation or affinity pairs may be envisaged to facilitate the association between the sdAb and the second molecule, the coupling may preferably involve one or more covalent bonds, i.e., chemical bonds that entail the sharing of one or more electron pairs between two atoms. In certain embodiments, the coupling may be direct, such that the chemical interactions or bond(s) occur between one or more atoms of the antibody and one or more atoms of the second molecule, whereas in other embodiments, the connection may be indirect, in particular via a suitable linker or spacer.

The nature and structure of such linkers is not particularly limited. The linker may be a rigid linker or a flexible linker. In particular embodiments, the linker is a covalent linker, achieving a covalent bond. A linker may be, for example, a (poly)peptide or non-peptide linker, such as a non-peptide polymer, such as a non-biological polymer. Preferably, any linkages may be hydrolytically stable linkages, i.e., substantially stable in water at useful pH values, including in particular under physiological conditions, for an extended period of time, e.g., for hours or days. When two or more linkers are used, these may be each independently the same or different.

In certain embodiments, a linker may be a stretch of between 1 and 50, such as between 1 and 40, between 1 and 30, or between 1 and 20 identical or non-identical units, wherein a unit is an amino acid, a monosaccharide, a nucleotide or a monomer. Non-identical units can be non-identical units of the same nature (e.g. different amino acids, or some copolymers). They can also be non-identical units of a different nature, e.g. a linker with amino acid and nucleotide units, or a heteropolymer (copolymer) comprising two or more different monomeric species. According to certain embodiments, a linker may be independently composed of 1 to 10 units of the same nature, particularly of 1 to 5 units of the same nature.

In particular embodiments, a linker may be a peptide or polypeptide linker of one or more amino acids. More particularly, the peptide linker may be 1 to 50 amino acids long, such as 1 to 40 or 1 to 30 amino acids long, preferably 1 to 20 amino acids long, such as more preferably 1 to 10 amino acids long. For example, the linker may be exactly 1, 2, 3, 4, 5, 6, 7, 8, 9, or 10 amino acids long. The nature of amino acids constituting the linker is not of particular relevance so long as the binding of the sdAb to CD38 and the desirable properties of the second molecule (e.g., detectability, anti-neoplastic impact on the antibody-bound cells) are not substantially impaired. Preferred linkers are essentially non-immunogenic and/or not prone to proteolytic cleavage. In certain embodiments, the linker may contain a predicted secondary structure such as an alpha-helical structure. However, linkers predicted to assume flexible, random coil structures are preferred. Linkers having tendency to form beta-strands may be less preferred or may need to be avoided. Cysteine residues may be less preferred or may need to be avoided due to their capacity to form intermolecular disulphide bridges. Basic or acidic amino acid residues, such as arginine, lysine, histidine, aspartic acid and glutamic acid may be less preferred or may need to be avoided due to their capacity for unintended electrostatic interactions. In certain preferred embodiments, the peptide linker may comprise, consist essentially of or consist of amino acids selected from the group consisting of glycine, serine, alanine, threonine, proline, and combinations thereof. In even more preferred embodiments, the peptide linker may comprise, consist essentially of or consist of amino acids selected from the group consisting of glycine, serine, and combinations thereof (glycine linkers, serine linkers, mixed glycine/serine linkers, glycine- and serine-rich linkers). In certain embodiments, the linker may consist essentially of or consist of glycine and serine residues. In certain embodiments, the peptide linker may consist of only glycine residues. In certain embodiments, the peptide linker may consist of only serine residues. Such linkers provide for particularly good flexibility.

In certain embodiments, the linker may be a non-peptide linker. In preferred embodiments, the non-peptide linker may comprise, consist essentially of or consist of a non-peptide polymer. The term “non-peptide polymer” as used herein refers to a biocompatible polymer including two or more repeating units linked to each other by a covalent bond excluding the peptide bond. For example, the non-peptide polymer may be 2 to 200 units long or 2 to 100 units long or 2 to 50 units long or 2 to 45 units long or 2 to 40 units long or 2 to 35 units long or 2 to 30 units long or 5 to 25 units long or 5 to 20 units long or 5 to 15 units long. The non-peptide polymer may be selected from the group consisting of polyethylene glycol, polypropylene glycol, copolymers of ethylene glycol and propylene glycol, polyoxyethylated polyols, polyvinyl alcohol, polysaccharides, dextran, polyvinyl ethyl ether, biodegradable polymers such as PLA (poly(lactic acid) and PLGA (polylactic-glycolic acid), lipid polymers, chitins, hyaluronic acid, and combinations thereof. Particularly preferred is poly(ethylene glycol) (PEG). Another particularly envisaged chemical linker is Ttds (4,7,10-trioxatridecan-13-succinamic acid). The molecular weight of the non-peptide polymer preferably may range from 1 to 100 kDa, and preferably 1 to 20 kDa. The non-peptide polymer may be one polymer or a combination of different types of polymers. The non-peptide polymer has reactive groups capable of binding to the elements which are to be coupled by the linker. Preferably, the non-peptide polymer has a reactive group at each end. Preferably, the reactive group is selected from the group consisting of a reactive aldehyde group, a propione aldehyde group, a butyl aldehyde group, a maleimide group and a succinimide derivative. The succinimide derivative may be succinimidyl propionate, hydroxy succinimidyl, succinimidyl carboxymethyl or succinimidyl carbonate. The reactive groups at both ends of the non-peptide polymer may be the same or different. In certain embodiments, the non-peptide polymer has a reactive aldehyde group at both ends. For example, the non-peptide polymer may possess a maleimide group at one end and, at the other end, an aldehyde group, a propionic aldehyde group or a butyl aldehyde group. When a polyethylene glycol (PEG) having a reactive hydroxy group at both ends thereof is used as the non-peptide polymer, the hydroxy group may be activated to various reactive groups by known chemical reactions, or a PEG having a commercially-available modified reactive group may be used so as to prepare the protein conjugate.

Any homo- or preferably heterobifunctional chemical crosslinking compound (or crosslinker) capable of crosslinking a protein (through a first reactive end of the crosslinker capable of forming a covalent bond with a functional group present in proteins, such as a primary amine or sulfhydryl group) and the second molecule (through a second reactive end of the crosslinker capable of forming a covalent bond with a functional group present in the second molecule) can be employed for the purposes of the present coupling. Non-limiting examples of homobifunctional crosslinkers include glutaraldehyde, diethylmalonimidate hydrochloride, and difluorodinitrophenylsulfonate. Non-limiting examples of heterobifunctional crosslinkers include m-maleimidobenzoyl-N-hydroxysuccinimide ester (MBS), succinimidyl-[(N-maleimidopropionamido)tetraethyleneglycol]ester (NHS-PEG4Mal), succinimidyl 3-(2-pyridyldithio)propionate (SPDP), and sulfosuccinimidyl 4-(N-maleimidomethyl) cyclohexane-1-carboxylate (Sulfo-SMCC).

In certain embodiments, the second molecule is detectable, whereby the antibody coupled therewith and the targets such as cells to which the antibody binds are endowed with a detectable character. The second molecule may thus provide a qualitative or preferably quantitative occurrence of or a change in a signal that is directly or indirectly observable either by visual observation or by instrumentation. The detectable character may be entailed by an atom, portion, functional group or moiety comprised by the second molecule. Detectable labels or entities of a variety of types exist, including without limitation dyes, radiolabels, electron-dense reagents, enzymes such as horse-radish peroxidase or luciferase, binding moieties such as biotin-streptavidin, haptens such as digoxigenin, luminogenic, phosphorescent or fluorogenic moieties, mass tags, fluorescent dyes optionally in combination with fluorescence resonance energy transfer (FRET) moieties, fluorescent proteins, etc. Such detectable labels may be suitably detectable by for example by mass spectrometric, spectroscopic, optical, photonic, electromagnetic, colourimetric, magnetic, photochemical, biochemical, immunochemical or chemical detection means, etc.

Preferably, as the disease monitoring/diagnostic applications envisaged herein may be primarily based on imaging methods to visualise and quantify the presence and distribution of CD38 positive neoplastic cells in a patient's body, the second molecule may be selected such as to be detectable when present in the body of the subject (in situ) after having been administered to the subject, so as to convey information on the bio-distribution of the antibody and thus of the cells bound thereby.

In certain embodiments, the second molecule may be a signal-emitting molecule. For example, the second molecule may emit a signal, such as a nuclear particle or electromagnetic radiation, which is directly detectable by (a set of) detectors positioned around the subject, or the molecule may emit a signal, such as a nuclear particle or electromagnetic radiation, which gets converted in situ into another signal, the latter signal being detectable by the (set of) detectors.

In certain preferred embodiments, the signal-emitting molecule may be detectable by positron emission tomography (PET) or by single photon emission computed tomography (SPECT). In the former case, the second molecule may emit positrons, e.g., the second molecule may comprise or consist of a radionuclide atom which decays by emission of a positron. The positron will annihilate with an electron in the tissue, releasing two 511 keV photons, which are detected by a PET scanner. In the latter case, the second molecule may emit gamma photons, e.g., the second molecule may comprise or consist of a radionuclide atom which decays by emission of gamma radiation, which are detected by a SPECT scanner.

Hence, in particularly preferred embodiments, the second molecule comprises, consists essentially of or consist of a radionuclide. The term “radionuclide” is used in line with its common meaning, denoting a nuclide which is radioactive, i.e., which displays the property of undergoing spontaneous nuclear transformation(s), preferably radioactive decay, with the emission of radiation, such as emission of subatomic particles and/or electromagnetic radiation, such as in particular alpha, beta, positron, neutron and/or gamma radiation. The term may be synonymous and interchangeable with terms such as “radioactive nuclide”, “radioisotope” or “radioactive isotope”, which are also commonplace in the art. Radionuclides occur naturally or can be produced artificially.

Without limitation, examples of radionuclides useful in imaging/diagnostic applications, such as PET or SPECT imaging, such as in particular gamma or positron emitters, include Technetium-99m, Indium-111, Rubidium-82, Thallium-201, Fluorine-18, Gallium-68, or Zirconium-89.

As mentioned, the second molecule can facilitate or can also facilitate therapy of a neoplastic disease, in particular it can have a cytotoxic effect on CD38-expressing neoplastic cells bound by the antibody, i.e., the second molecule may be cytotoxic. Cancerous growths tend to be particularly sensitive to damage by radiation. In certain embodiments, where the second molecule comprises, consist essentially of or consists of a radionuclide, such radionuclide may be cytotoxic. Preferably, the radionuclide may display a degree of toxicity to cells bound by the antibody which is proportional to the level of CD38 expression by the cells. In certain embodiments, radioisotopes employed in radiopharmaceuticals may be particularly beta or alpha particle emitters. Without limitation, examples of radionuclides useful in radiopharmaceuticals include Lutetium-177, Yttrium-90, Iodine-131, Samarium-153, Phosphorus-32, Bismuth-213, Lead-212, Radium-223, Thorium-227, Actinium-225, or Astatine-211. Particularly preferred may be Lu-177.

Certain radionuclides may facilitate both the detection (imaging/diagnosis) of CD38+ cells and a cytotoxic effect thereupon. By means of an example, Lu-177 is a strong beta emitter, which facilitates cytotoxicity, capable with enough gamma emission to enable SPECT imaging. In other embodiments, the decoration of the antibody with two distinct radionuclides to enable both detection and cytotoxicity can be envisaged.

Chemistries to incorporate radionuclides in protein-based imaging agents or radiopharmaceuticals such as antibodies are generally known and are also exemplified in non-limiting manner in the experimental section. Further guidance can be found in publications such as Mather S. J. (1986) Radiolabelled Antibodies as Radiopharmaceuticals. In: Cox P. H., Mather S. J., Sampson C. B., Lazarus C. R. (eds) Progress in Radiopharmacy. Developments in Nuclear Medicine, vol 10. Springer, Dordrecht; Saha G. B. (1992) Radiopharmaceuticals and Methods of Radiolabeling. In: Fundamentals of Nuclear Pharmacy. Springer, New York, N.Y.; and Aluicio-Sarduy et al. PET radiometals for antibody labeling. J Labelled Comp Radiopharm. 2018, vol. 61(9), 636-651.

In certain aspects, the anti-CD38 single-domain antibody is for use in a method of diagnosis or monitoring a neoplastic disease in a subject. In certain aspects, the anti-CD38 single-domain antibody is for use in a method of diagnosis or monitoring, and treating a neoplastic disease in a subject.

In certain embodiments, the present anti-CD38 single-domain antibody may be employed in so-called pre-targeting strategies useful in methods of diagnosis, monitoring, and/or therapy of neoplastic diseases, such as explained in more detail in the experimental section. In particular, the fact that the complex between CD38 and the present anti-CD38 sdAb bound thereto is not or is only minimally internalised by the cells means that the unlabelled anti-CD38 sdAb can be administered first, allowed to bind to CD38⁺ target cells even while the excess anti-CD38 sdAb is removed from circulation, followed by the administration of a second agent, which is capable of specifically binding to the anti-CD38 sdAb and which comprises the second molecule as intended herein, such as a radionuclide, which facilitates the imaging and/or therapy. Hence, the second agent may comprise, consist essentially of, or consist of a) a part capable of specifically binding to the anti-CD38 sdAb and b) the second molecule. Accordingly, the indirect coupling between the anti-CD38 sdAb and the second molecule can be seen as arising once the second agent recognises and specifically binds to the anti-CD38 sdAb. Specific binding between the anti-CD38 sdAb and the second agent can be effected by any means known in the art, such as by incorporating one component of a specific binding pair (affinity pair) in the anti-CD38 sdAb and the other component of the specific binding pair in the second agent. Specific binding pairs include, without limitation, biotin-avidin or biotin-streptavidin binding pairs, complementary oligonucleotide pairs, and complementary peptide nucleic acid (PNA) oligonucleotide pairs. Another option is to provide the anti-CD38 sdAb as a bispecific antibody also comprising an arm specifically binding to the second agent, such as to a radiolabeled chelator. Accordingly, provided is also a combination or kit of parts comprising unlabeled anti-CD38 sdAb as taught herein and a second agent capable of specifically binding to the anti-CD38 sdAb and comprising the second molecule as intended herein. Pre-targeting can advantageously reduce exposure of non-target tissues to the second molecule, such as a radionuclide, such as more particularly a cytotoxic radionuclide.

The term “neoplastic disease” generally refers to any disease or disorder characterised by neoplastic cell growth and proliferation, whether benign (not invading surrounding normal tissues, not forming metastases), pre-malignant (pre-cancerous), or malignant (invading adjacent tissues and capable of producing metastases). The term neoplastic disease generally includes all transformed cells and tissues and all cancerous cells and tissues. Neoplastic diseases or disorders include, but are not limited to abnormal cell growth, benign tumors, premalignant or precancerous lesions, malignant tumors, and cancer. Examples of neoplastic diseases or disorders are benign, pre-malignant, or malignant neoplasms located in any tissue or organ, such as in the prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, blood, pelvic, skin, soft tissue, spleen, thoracic, or urogenital tract.

As used herein, the terms “tumor” or “tumor tissue” refer to an abnormal mass of tissue that results from excessive cell division. A tumor or tumor tissue comprises tumor cells which are neoplastic cells with abnormal growth properties and no useful bodily function. Tumors, tumor tissue and tumor cells may be benign, pre-malignant or malignant, or may represent a lesion without any cancerous potential. A tumor or tumor tissue may also comprise tumor-associated non-tumor cells, e.g., vascular cells which form blood vessels to supply the tumor or tumor tissue. Non-tumor cells may be induced to replicate and develop by tumor cells, for example, the induction of angiogenesis in a tumor or tumor tissue.

As used herein, the term “cancer” refers to a malignant neoplasm characterised by deregulated or unregulated cell growth. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). The term “metastatic” or “metastasis” generally refers to the spread of a cancer from one organ or tissue to another non-adjacent organ or tissue. The occurrence of the neoplastic disease in the other non-adjacent organ or tissue is referred to as metastasis.

Examples of cancer include but are not limited to carcinoma, lymphoma, blastoma, sarcoma, and leukemia or lymphoid malignancies.

The present antibody agents and methods are intended to evaluate and target neoplastic diseases such as cancers characterised by an increased expression of CD38 compared to normal, healthy cells or tissues. As mentioned earlier, CD38 is a surface antigen which, while being ubiquitously expressed in many cells, especially in plasma cells and other lymphoid and myeloid cell populations, is differentially very highly and uniformly expressed in several malignancies, in particular in hematologic malignancies, including inter alia multiple myeloma (MM), non-hodgkin lymphoma (NHL) and chronic lymphoid leukemia (CLL), and has been proven to be a good target for detection and immunotherapy of such diseases.

Accordingly, in certain embodiments, the neoplastic disease comprises neoplastic cells expressing CD38 antigen at the cell surface. In certain embodiments, the surface expression of CD38 on neoplastic cells may be at least 3×higher, or at least 5×higher, or at least 10×higher, or at least 50×higher, or at least 100×higher, or at least 500×higher, or at least 1000×higher than CD38 expression on normal or healthy cells of the corresponding cell type or tissue. Such ratios may be suitably determined by comparing arithmetic means of CD38 quantity as determined by any suitable method in the respective cell populations.

In certain embodiments, the tumor is a solid tumor. Solid tumors encompass any tumors forming a neoplastic mass that usually does not contain cysts or liquid areas. Solid tumors may be benign, pre-malignant or malignant. Examples of solid tumors are carcinomas, sarcomas, melanomas and lymphomas. Solid tumors also encompass metastases originated from solid tumors. In certain embodiments, the tumor, such as a solid tumor, including any metastases of the tumor, such as any metastases of a solid tumor, may be of epithelial, mesenchymal or melanocyte origin. In certain embodiments, the tumor may be a carcinoma, including any malignant neoplasm originated from epithelial tissue in any of several sites, such as without limitation skin, lung, intestine, colon, breast, bladder, head and neck (including lips, oral cavity, salivary glands, nasal cavity, nasopharynx, paranasal sinuses, pharynx, throat, larynx, and associated structures), esophagus, thyroid, kidney, liver, pancreas, bladder, penis, testes, prostate, vagina, cervix, or anus. In certain embodiments, the tumor may be a sarcoma, including any malignant neoplasm originated from mesenchymal tissue in any of several sites, such as without limitation bone, cartilage, fat, muscle, blood vessels, fibrous tissue, or other connective or supportive tissue. In certain embodiments, the tumor may be a melanoma, including any malignant neoplasm originated from melanocytes in any of several sites, such as without limitation skin, mouth, eyes, or small intestine. In certain embodiments, the tumor, such as a solid tumor, is hepatocellular carcinoma, lung cancer, melanoma, breast cancer or glioma.

In certain embodiments, the neoplastic disease is a tumor affecting the blood, bone marrow, lymph, and/or lymphatic system. This encompasses malignancies deriving from the myeloid cell lineage as well as from the lymphoid cell lineage. Lymphomas, lymphocytic leukemias, and myeloma, as well as acute and chronic myelogenous leukemia, myelodysplastic syndromes and myeloproliferative diseases are encompassed.

In certain embodiments, the neoplastic disease is a hematological malignancy (blood cancer), including leukemias, lymphomas, and myelomas, and more particularly including Acute lymphoblastic leukemia (ALL), Acute myelogenous leukemia (AML), Chronic lymphocytic leukemia (CLL), Chronic myelogenous leukemia (CML), Acute monocytic leukemia, other leukemias, Hodgkin's lymphomas, Non-Hodgkin's lymphomas, and myelomas.

In certain embodiments, the neoplastic disease is multiple myeloma (plasma cell myeloma), a plasma-cell cancer characterised by accumulation of malignant cells in the bone marrow and production of a monoclonal immunoglobulin (M protein).

The term “diagnosis” is commonplace and well-understood in medical practice. By means of further explanation and without limitation the term “diagnosis”, or its alternative forms such as “diagnosing”, generally refers to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition). The term “monitoring” generally refers to the follow-up of a disease or a condition in a subject for any changes which may occur over time.

The terms “subject”, “individual” or “patient” are used interchangeably throughout this specification, and typically and preferably denote humans, but may also encompass reference to non-human animals, preferably warm-blooded animals, even more preferably non-human mammals. Particularly preferred are human subjects including both genders and all age categories thereof. In other embodiments, the subject is an experimental animal or animal substitute as a disease model. The term does not denote a particular age or sex. Thus, adult and newborn subjects, as well as fetuses, whether male or female, are intended to be covered. The term subject is further intended to include transgenic non-human species.

The term “subject in need of treatment” or similar as used herein refers to subjects diagnosed with or having a disease as recited herein and/or those in whom said disease is to be prevented.

Reference to “therapy” or “treatment” broadly encompasses both curative and preventative treatments, and the terms may particularly refer to the alleviation or measurable lessening of one or more symptoms or measurable markers of a pathological condition such as a disease or disorder. The terms encompass primary treatments as well as neo-adjuvant treatments, adjuvant treatments and adjunctive therapies. Measurable lessening includes any statistically significant decline in a measurable marker or symptom. Generally, the terms encompass both curative treatments and treatments directed to reduce symptoms and/or slow progression of the disease. The terms encompass both the therapeutic treatment of an already developed pathological condition, as well as prophylactic or preventative measures, wherein the aim is to prevent or lessen the chances of incidence of a pathological condition. In certain embodiments, the terms may relate to therapeutic treatments. In certain other embodiments, the terms may relate to preventative treatments. Treatment of a chronic pathological condition during the period of remission may also be deemed to constitute a therapeutic treatment. The term may encompass ex vivo or in vivo treatments as appropriate in the context of the present invention.

The term “therapeutically effective amount” generally denotes an amount sufficient to elicit the pharmacological effect or medicinal response in a subject that is being sought by a medical practitioner such as a medical doctor, clinician, surgeon, veterinarian, or researcher, which may include inter alia alleviation of the symptoms of the disease being treated, in either a single or multiple doses. Appropriate therapeutically effective doses of the present molecules may be determined by a qualified physician with due regard to the nature and severity of the disease, and the age and condition of the patient. The effective amount of the molecules described herein to be administered can depend on many different factors and can be determined by one of ordinary skill in the art through routine experimentation. Several non-limiting factors that might be considered include biological activity of the active ingredient, nature of the active ingredient, characteristics of the subject to be treated, etc. The term “to administer” generally means to dispense or to apply, and typically includes both in vivo administration and ex vivo administration to a tissue, preferably in vivo administration. Generally, compositions may be administered systemically or locally.

In certain embodiments, the subject may have been selected as having or suspected of having the neoplastic disease as discussed herein, such as a hematological malignancy, such as more particularly multiple myeloma. Any diagnostic methodology suitable for arriving at such characterisation of the patient may be employed, including consideration of symptoms, sample (e.g., biopsy, aspirate) cytology or histology, blood count, blood film, karyotype, DNA-, RNA- and/or protein-based molecular markers, biochemical or metabolic markers, medical imaging (e.g., computer tomography, magnetic resonance imaging), etc.

In certain embodiments, the diagnostic or monitoring applications of the anti-CD38 sdAbs may involve medical imaging. In such embodiments, the second molecule or label coupled with the antibody is thus detectable by an imaging modality, allowing to visualise the CD38 expressing tumor or cancer cells to which the anti-CD38 antibody has specifically bound in the subject. The bio-distribution and concentration of the antibody thus reveals the presence, location and/or amount of CD38-expressing cells.

The term “imaging” broadly encompasses any medical imaging technique or process for creating visual representations of the interior of a body and/or visual representation of the function of organs or tissues. Imaging modalities or technologies as envisaged herein may include but are not limited to X-ray radiography, X-ray computed tomography (CT), magnetic resonance imaging (MRI), positron emission tomography (PET), PET-CT, and single-photon emission computed tomography (SPECT). Preferably, the imaging modality may be PET, PET-CT, or SPECT.

Hence, an aspect provides an imaging method for monitoring the presence, location and/or amount of CD38-expressing cells in a subject comprising the steps of:
i) detecting, in a subject to whom a detectable quantity of an anti-CD38 single-domain antibody, preferably an antibody as discussed above, directly or indirectly coupled to a signal-emitting molecule has been administered, signal emitted by said signal-emitting molecule coupled to said antibody; and
ii) generating an image representative of the location and/or quantity or intensity of said signal.

Preferably, the emitted signal may be detected by positron emission tomography (PET) and a PET image is generated. Or preferably, the emitted signal is detected by single photon emission computed tomography (SPECT) and a SPECT image is generated.

In certain embodiments, the method may further comprise a step of superimposing the PET or SPECT image with at least one computed tomography (CT) scan or at least one magnetic resonance image (MRI).

A patient subjected to such an imaging method may in certain embodiments have or be suspected of having or be under treatment for a neoplastic disease, such as a hematological malignancy, more particularly multiple myeloma, as detailed elsewhere in this specification.

In certain embodiments, the imaging method may be used to monitor, follow-up or track the progression of the neoplastic disease over time by generating images that lend themselves to a side-by-side comparison (e.g., images generated with the same quantity of the antibody per kg subject weight and the same route and manner of administration; using substantially the same settings on the imaging system; etc.) at two or more sequential time points, optionally where the patient has received or may be receiving an anti-neoplastic therapy.

Accordingly, in certain embodiments, the method comprises conducting step i) on at least two distinct time points. Preferably a first time point may be prior to the start of a given therapy. Preferably, a second, subsequent time point may be during or after the therapy. For example, the scans may be scheduled before and after a change in the type and/or dosage regiment of a therapy. For example, the scans may be scheduled before and after a change in the patient's subjective condition or objective clinical picture. For example, the scans may be scheduled at substantially regular intervals during or after the therapy, for example to monitor cancer regression, remission or relapse. Other appropriate applications of the imaging methods described herein will be apparent to the skilled person.

In certain embodiments, the two or more distinct signal-emitting molecules may be detected in the imaging method. This may for example allow for detection and visualisation of multiple targets, cell types, tissues etc. Hence, in certain embodiments, the method may further comprise detecting at least one additional signal-emitting molecule, preferably wherein said additional signal-emitting molecule is coupled to an affinity ligand (such as an immunological affinity agent, such as an antibody) capable of binding a target molecule different from CD38.

The antibodies as taught herein can be formulated into pharmaceutical compositions. Therefore, any reference to the use of the antibodies in diagnosis, monitoring, therapy or imaging (or any variation of such language) also subsumes such uses of pharmaceutical compositions comprising the antibodies. The terms “pharmaceutical composition” and “pharmaceutical formulation” may be used interchangeably. The pharmaceutical compositions as taught herein may comprise in addition to the one or more actives (antibodies), one or more pharmaceutically or acceptable carriers. Suitable pharmaceutical excipients depend on the dosage form and identities of the active ingredients and can be selected by the skilled person (e.g., by reference to the Handbook of Pharmaceutical Excipients 7^{th} Edition 2012, eds. Rowe et al.).

As used herein, the terms “carrier” or “excipient” are used interchangeably and broadly include any and all solvents, diluents, buffers (such as, e.g., neutral buffered saline, phosphate buffered saline, or optionally Tris-HCl, acetate or phosphate buffers), solubilisers (such as, e.g., Tween® 80, Polysorbate 80), colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives (such as, e.g., Thimerosal™, benzyl alcohol), antioxidants (such as, e.g., ascorbic acid, sodium metabisulfite), tonicity controlling agents, absorption delaying agents, adjuvants, bulking agents (such as, e.g., lactose, mannitol) and the like. The use of such media and agents for the formulation of pharmaceutical compositions is well known in the art. Acceptable diluents, carriers and excipients typically do not adversely affect a recipient's homeostasis (e.g., electrolyte balance). The use of such media and agents for pharmaceutical active substances is well known in the art. Such materials should be non-toxic and should not interfere with the activity of the actives. Acceptable carriers may include biocompatible, inert or bioabsorbable salts, buffering agents, oligo- or polysaccharides, polymers, viscosity-improving agents, preservatives and the like. One exemplary carrier is physiologic saline (0.15 M NaCl, pH 7.0 to 7.4). Another exemplary carrier is 50 mM sodium phosphate, 100 mM sodium chloride.

The precise nature of the carrier or other material will depend on the route of administration. For example, the pharmaceutical composition may be in the form of a parenterally acceptable aqueous solution, which is pyrogen-free and has suitable pH, isotonicity and stability. Preferably, the pH value of the pharmaceutical formulation is in the physiological pH range, such as particularly the pH of the formulation is between about 5 and about 9.5, more preferably between about 6 and about 8.5, even more preferably between about 7 and about 7.5.

While pharmaceutical compositions as intended herein may be formulated for essentially any route of administration, parenteral administration (such as, e.g., subcutaneous, intravenous (I.V.), intramuscular, intraperitoneal or intrasternal injection or infusion) or topical administration may be preferred. The effects attainable can be, for example, systemic, local, tissue-specific, etc., depending of the specific needs of a given application. In certain embodiments, an I.V. bolus injection or infusion may advantageously allow the antibody to enter circulation and be distributed throughout the body, allowing to label CD38⁺ expressing cells and tissues.

One skilled in this art will recognise that the above description is illustrative rather than exhaustive. Indeed, many additional formulations techniques and pharmaceutically-acceptable excipients and carrier solutions are well-known to those skilled in the art, as is the development of suitable dosing and treatment regimens for using the particular compositions described herein in a variety of administration or treatment regimens.

The dosage or amount of the antibodies as taught herein, optionally in combination with one or more other active compounds to be administered, depends on the individual case and is, as is customary, to be adapted to the individual circumstances to achieve an optimum effect. Thus, the unit dose and regimen depend on the nature and the severity of the disorder to be treated, and also on factors such as the species of the subject, the sex, age, body weight, general health, diet, mode and time of administration, immune status, and individual responsiveness of the human or animal to be treated, efficacy, metabolic stability and duration of action of the compounds used, on whether the therapy is acute or chronic or prophylactic, or on whether other active compounds are administered in addition to the agent of the invention.

Without limitation, depending on the type and severity of the disease, a typical dosage (e.g., a typical daily dosage or a typical intermittent dosage, e.g., a typical dosage for every two days, every three days, every four days, every five days, every six days, every week, every 1.5 weeks, every two weeks, every three weeks, every month, or other) of the molecules as taught herein may range from about 10 μg/kg to about 100 mg/kg body weight of the subject, per dose, depending on the factors mentioned above, e.g., may range from about 100 μg/kg to about 100 mg/kg body weight of the subject, per dose, or from about 200 μg/kg to about 75 mg/kg body weight of the subject, per dose, or from about 500 μg/kg to about 50 mg/kg body weight of the subject, per dose, or from about 1 mg/kg to about 25 mg/kg body weight of the subject, per dose, or from about 1 mg/kg to about 10 mg/kg body weight of the subject, per dose, e.g., may be about 100 μg/kg, about 200 μg/kg, about 300 μg/kg, about 400 μg/kg, about 500 μg/kg, about 600 μg/kg, about 700 μg/kg, about 800 μg/kg, about 900 μg/kg, about 1.0 mg/kg, about 2.0 mg/kg, about 5.0 mg/kg, about 10 mg/kg, about 15 mg/kg, about 20 mg/kg, about 30 mg/kg, about 40 mg/kg, about 50 mg/kg, about 75 mg/kg, or about 100 mg/kg body weight of the subject, per dose.

In certain embodiments, where the antibody comprises a radionuclide at taught herein, the radiochemical purity (the proportion of the total radioactvity in the sample which is present as the desired radiolabelled species, i.e., the radiolabelled antibody) of the sample may be at least 80%, preferably at least 85%, more preferably at least 90%, still more preferably at least 95%, yet more preferably at least 96%, at least 97%, at least 98%, and very preferably at least 99% or even 100%.

In certain embodiments, where the antibody comprises a radionuclide at taught herein, depending on the radionuclide and the purpose of administration (e.g., imaging, therapy), the administered dose may be between 10 MBq and 1000 MBq, such as between 50 MBq and 500 MBq, such as for example about 100 MBq, about 200 MBq, about 300 MBq, or about 400 MBq, such as preferably between about 50 MBq and 150 Mbq, such as about 75 MBq, about 100 MBq, or about 150 MBq. Such doses may for example be preferred for ¹¹¹In-labelled antibodies. In certain other embodiments, depending on the radionuclide and the purpose of administration, the administered dose may be between 1 and 50 MBq/kg, such as between 5 and 25 MBq/kg, such as about 10 MBq/kg, about 15 MBq/kg, or about 20 MBq/kg. Such doses may for example be preferred for ¹⁷⁷Lu-labelled antibodies. Such dose may typically be a one time dose, and may be followed by detection of the antibody by an imaging modality.

In certain embodiments, to minimise non-specific renal uptake of the present antibodies, e.g., to avoid a reduction of the antibody signal in other places and to minimise exposure of the kidneys to radioactivity, the antibodies may be co-administered, such as co-infused with a plasma-expander, such as human albumin, HMW dextran, hetastarch, hydroxyethyl starch, or gelatine solutions, at quantities customary in the art.

In certain embodiments, an antibody molecule as taught herein may be administered as the sole imaging and/or therapeutic agent, or in combination with one or more other pharmaceutical agents where the combination causes no unacceptable adverse effects. By means of an example, two or more antibodies as taught herein may be co-administered. By means of another example, one or more antibody as taught herein may be co-administered with a pharmaceutical agent that is not an antibody as envisaged herein. For example, the antibodies as taught herein may be combined with known anti-cancer therapy or therapies, such as for example surgery, radiotherapy, chemotherapy, biological therapy, or combinations thereof. The term “chemotherapy” as used herein is conceived broadly and generally encompasses treatments using chemical substances or compositions.

Chemotherapeutic agents may typically display cytotoxic or cytostatic effects. In certain embodiments, a chemotherapeutic agent may be an alkylating agent, a cytotoxic compound, an anti-metabolite, a plant alkaloid, a terpenoid, a topoisomerase inhibitor, or a combination thereof. The term “biological therapy” as used herein is conceived broadly and generally encompasses treatments using biological substances or compositions, such as biomolecules, or biological agents, such as viruses or cells. In certain embodiments, a biomolecule may be a peptide, polypeptide, protein, nucleic acid, or a small molecule (such as primary metabolite, secondary metabolite, or natural product), or a combination thereof. Examples of suitable biomolecules include without limitation interleukins, cytokines, anti-cytokines, tumor necrosis factor (TNF), cytokine receptors, vaccines, interferons, enzymes, therapeutic antibodies, antibody fragments, antibody-like protein scaffolds, or combinations thereof. Examples of suitable biomolecules include but are not limited to aldesleukine, alemtuzumab, atezolizumab, bevacizumab, blinatumomab, brentuximab vedotine, catumaxomab, cetuximab, daratumumab, denileukin diftitox, denosumab, dinutuximab, elotuzumab, gemtuzumab ozogamicin, ⁹⁰Y-ibritumomab tiuxetan, idarucizumab, interferon A, ipilimumab, necitumumab, nivolumab, obinutuzumab, ofatumumab, olaratumab, panitumumab, pembrolizumab, ramucirumab, rituximab, tasonermin, ¹³¹I-tositumomab, trastuzumab, Ado-trastuzumab emtansine, and combinations thereof. Examples of suitable oncolytic viruses include but are not limited to talimogene laherparepvec. Further categories of anti-cancer therapy include inter alia hormone therapy (endocrine therapy), immunotherapy, and stem cell therapy, which are commonly considered as subsumed within biological therapies. Examples of suitable hormone therapies include but are not limited to tamoxifen; aromatase inhibitors, such as atanastrozole, exemestane, letrozole, and combinations thereof; luteinizing hormone blockers such as goserelin, leuprorelin, triptorelin, and combinations thereof; anti-androgens, such as bicalutamide, cyproterone acetate, flutamide, and combinations thereof, gonadotrophin releasing hormone blockers, such as degarelix; progesterone treatments, such as medroxyprogesterone acetate, megestrol, and combinations thereof, and combinations thereof. The term “immunotherapy” broadly encompasses any treatment that modulates a subject's immune system. In particular, the term comprises any treatment that modulates an immune response, such as a humoral immune response, a cell-mediated immune response, or both. Immunotherapy comprises cell-based immunotherapy in which immune cells, such as T cells and/or dendritic cells, are transferred into the patient. The term also comprises an administration of substances or compositions, such as chemical compounds and/or biomolecules (e.g., antibodies, antigens, interleukins, cytokines, or combinations thereof), that modulate a subject's immune system. Examples of cancer immunotherapy include without limitation treatments employing monoclonal antibodies, for example Fc-engineered monoclonal antibodies against proteins expressed by tumor cells, immune checkpoint inhibitors, prophylactic or therapeutic cancer vaccines, adoptive cell therapy, and combinations thereof. Examples of immune checkpoint targets for inhibition include without limitation PD-1 (examples of PD-1 inhibitors include without limitation pembrolizumab, nivolumab, and combinations thereof), CTLA-4 (examples of CTLA-4 inhibitors include without limitation ipilimumab, tremelimumab, and combinations thereof), PD-L1 (examples of PD-L1 inhibitors include without limitation atezolizumab), LAG3, B7-H3 (CD276), B7-H4, TIM-3, BTLA, A2aR, killer cell immunoglobulin-like receptors (KIRs), IDO, and combinations thereof. Another approach to therapeutic anti-cancer vaccination includes dendritic cell vaccines. The term broadly encompasses vaccines comprising dendritic cells which are loaded with antigen(s) against which an immune reaction is desired. Adoptive cell therapy (ACT) can refer to the transfer of cells, most commonly immune-derived cells, such as in particular cytotoxic T cells (CTLs), back into the same patient or into a new recipient host with the goal of transferring the immunologic functionality and characteristics into the new host. If possible, use of autologous cells helps the recipient by minimizing tissue rejection and graft vs. host disease issues. Various strategies may for example be employed to genetically modify T cells by altering the specificity of the T cell receptor (TCR) for example by introducing new TCR α and β chains with selected peptide specificity. Alternatively, chimeric antigen receptors (CARs) may be used in order to generate immunoresponsive cells, such as T cells, specific for selected targets, such as malignant cells, with a wide variety of receptor chimera constructs having been described. Examples of CAR constructs include without limitation 1) CARs consisting of a single-chain variable fragment of an antibody specific for an antigen, for example comprising a V_{L} linked to a V_{H} of a specific antibody, linked by a flexible linker, for example by a CD8α hinge domain and a CD8α transmembrane domain, to the transmembrane and intracellular signaling domains of either CD3ζ or FcRγ; and 2) CARs further incorporating the intracellular domains of one or more costimulatory molecules, such as CD28, OX40 (CD134), or 4-1BB (CD137) within the endodomain, or even including combinations of such costimulatory endodomains. Stem cell therapies in cancer commonly aim to replace bone marrow stem cells destroyed by radiation therapy and/or chemotherapy, and include without limitation autologous, syngeneic, or allogeneic stem cell transplantation. The stem cells, in particular hematopoietic stem cells, are typically obtained from bone marrow, peripheral blood or umbilical cord blood. Details of administration routes, doses, and treatment regimens of anti-cancer agents are known in the art, for example as described in “Cancer Clinical Pharmacology” (2005) ed. By Jan H. M. Schellens, Howard L. McLeod and David R. Newell, Oxford University Press. Active components of any combination therapy may be admixed or may be physically separated, and may be administered simultaneously or sequentially in any order.

In a particularly preferred embodiment, the present antibodies, as imaging and/or therapeutic actives, may be combined with one or more anti-neoplastic agents targeting CD38 expressing neoplastic cells, such as with one or more therapeutic anti-CD38 antibodies, such as preferably with daratumumab (Darzalex®) and/or isatuximab (Sarclisa®) at a customary dosage. In an illustrative embodiment, an antibody as taught herein may be employed as an imaging agent, and where the imaging method as interpreted by a radiologist or a clinician indicates that a patient would benefit from an anti-CD38 treatment (e.g., due to the presence of a predetermined quantity of signal attributable to CD38+ neoplastic cells), such treatment (e.g., with daratumumab and/or isatuximab) may be administered. In another illustrative embodiment, an antibody as taught herein may be employed as an imaging and therapeutic (theranostic) agent, and where the imaging method as interpreted by a radiologist or a clinician indicates that a patient would benefit from an anti-CD38 treatment, such treatment (e.g., with daratumumab and/or isatuximab) may be administered as a combination therapy.

The present application also provides aspects and embodiments as set forth in the following Statements:

Statement 1. An anti-CD38 single-domain antibody directly or indirectly coupled to a second molecule for use in a method of diagnosis or monitoring a neoplastic disease in a subject, or for use in a method of treating a neoplastic disease in a subject, wherein the antibody comprises an amino acid sequence that comprises 3 complementary determining regions (CDR1 to CDR3);

wherein CDR1 is chosen from the group consisting of:
a)

(SEQ ID NO: 1)
YTDSDYI,

b) Polypeptides that have at least 80% amino acid sequence identity with SEQ ID NO: 1,
c) Polypeptides that have 3, 2 or 1 amino acid difference with SEQ ID NO: 1,

wherein CDR2 is chosen from the group consisting of:
a)

(SEQ ID NO: 2)
TIYIGGTYIH,

b) Polypeptides that have at least 80% amino acid sequence identity with SEQ ID NO: 2,
c) Polypeptides that have 3, 2 or 1 amino acid difference with SEQ ID NO: 2,

and wherein CDR3 is chosen from the group consisting of:
a)

(SEQ ID NO: 3)
AATKWRPFISTRAAEYNY,

b) Polypeptides that have at least 80% amino acid sequence identity with SEQ ID NO: 3,
c) Polypeptides that have 3, 2 or 1 amino acid difference with SEQ ID NO: 3.

Statement 2. An anti-CD38 single-domain antibody directly or indirectly coupled to a second molecule for use in a method of diagnosis or monitoring and treating a neoplastic disease in a subject, wherein the antibody comprises an amino acid sequence that comprises 3 complementary determining regions (CDR1 to CDR3);

wherein CDR1 is chosen from the group consisting of:
a)

(SEQ ID NO: 1)
YTDSDYI,

b) Polypeptides that have at least 80% amino acid sequence identity with SEQ ID NO: 1,
c) Polypeptides that have 3, 2 or 1 amino acid difference with SEQ ID NO: 1,

wherein CDR2 is chosen from the group consisting of:
a)

(SEQ ID NO: 2)
TIYIGGTYIH,

b) Polypeptides that have at least 80% amino acid sequence identity with SEQ ID NO: 2,
c) Polypeptides that have 3, 2 or 1 amino acid difference with SEQ ID NO: 2,

and wherein CDR3 is chosen from the group consisting of:
a)

(SEQ ID NO: 3)
AATKWRPFISTRAAEYNY,

b) Polypeptides that have at least 80% amino acid sequence identity with SEQ ID NO: 3,
c) Polypeptides that have 3, 2 or 1 amino acid difference with SEQ ID NO: 3.

Statement 3. The anti-CD38 single-domain antibody directly or indirectly coupled to the second molecule for use according to Statements 1 or 2, wherein the amino acid sequence of CDR1 is YTDSDYI (SEQ ID NO: 1), the amino acid sequence of CDR2 is TIYIGGTYIH (SEQ ID NO: 2), and the amino acid sequence of CDR3 is AATKWRPFISTRAAEYNY (SEQ ID NO: 3).

Statement 4. The anti-CD38 single-domain antibody directly or indirectly coupled to the second molecule for use according to any one of Statements 1 to 3, wherein said antibody is a heavy chain variable domain derived from a heavy-chain antibody (V_{HH}) or a functional fragment thereof.

Statement 5. The anti-CD38 single-domain antibody directly or indirectly coupled to the second molecule for use according to any one of Statements 1 to 4, wherein said antibody comprises, consists essentially of or consists of an amino acid sequence having a sequence identity of at least 80%, preferably at least 90%, more preferably at least 95%, even more preferably at least 99% to SEQ ID NO: 4 or a functional fragment thereof:

(SEQ ID NO: 4)
QVQLVESGGGSVQAGGSLRLSCAASGYTDSDYIMAWF
RQAPGKEREVVATIYIGGTYIHYADSVKGRFTISRDN
AENTVYLQMNNLKPEDTAMYYCAATKWRPFISTRAAE
YNYWGQGTLVTVSS.

Statement 6. The anti-CD38 single-domain antibody directly or indirectly coupled to the second molecule for use according to any one of Statements 1 to 5, wherein:
the neoplastic disease comprises a neoplastic cell expressing CD38 antigen at the cell surface;
the neoplastic disease is a solid tumor;
the neoplastic disease is hepatocellular carcinoma, lung cancer, melanoma, breast cancer or glioma;
the neoplastic disease is a hematological malignancy;
the neoplastic disease is multiple myeloma (MM), non-hodgkin lymphoma (NHL) or chronic lymphoid leukemia (CLL); and/or
the neoplastic disease is multiple myeloma.

Statement 7. The anti-CD38 single-domain antibody directly or indirectly coupled to the second molecule for use according to any one of Statements 1 to 6, wherein the second molecule is detectable, or cytotoxic, or detectable and cytotoxic.

Statement 8. The anti-CD38 single-domain antibody directly or indirectly coupled to the second molecule for use according to any one of Statements 1 to 7, wherein the second molecule is a signal-emitting molecule, preferably a signal-emitting molecule detectable by positron emission tomography (PET) or single photon emission computed tomography (SPECT), more preferably the second molecule comprises, consists essentially of or consist of a radionuclide.

Statement 9. The anti-CD38 single-domain antibody directly or indirectly coupled to the second molecule for use according to Statement 8, wherein the radionuclide is cytotoxic to cells bound by said antibody, preferably wherein the degree of toxicity is proportional to the level of CD38 expression by the cells.

Statement 10. The anti-CD38 single-domain antibody directly or indirectly coupled to the second molecule for use according to any one of Statements 1 to 9, wherein the method further comprises treating the subject with daratumumab.

Statement 11. The anti-CD38 single-domain antibody directly or indirectly coupled to the second molecule for use according to any one of Statements 1 to 10, wherein the method comprises administration of the unlabelled anti-CD38 sdAb, followed by administration of a second agent, which is capable of specifically binding to the anti-CD38 sdAb and which comprises the second molecule.

Statement 12. The anti-CD38 single-domain antibody directly or indirectly coupled to the second molecule for use according to any one of Statements 1 to 11, wherein the subject has been selected as having or suspected of having the neoplastic disease, such as a neoplastic disease as individualised in Statement 6, preferably a hematological malignancy, more preferably multiple myeloma.

Statement 13. An imaging method for evaluating or monitoring the presence, location and/or amount of CD38-expressing cells in a subject comprising the steps of:
i) detecting, in a subject to whom a detectable quantity of an anti-CD38 single-domain antibody directly or indirectly coupled to a signal-emitting molecule has been administered, signal emitted by said signal-emitting molecule coupled to said antibody; and
ii) generating an image representative of the location and/or quantity or intensity of said signal.

Statement 14. The method according to Statement 13, wherein the antibody comprises an amino acid sequence that comprises 3 complementary determining regions (CDR1 to CDR3);

wherein CDR1 is chosen from the group consisting of:
a)

(SEQ ID NO: 1)
YTDSDYI,

b) Polypeptides that have at least 80% amino acid sequence identity with SEQ ID NO: 1,
c) Polypeptides that have 3, 2 or 1 amino acid difference with SEQ ID NO: 1,

wherein CDR2 is chosen from the group consisting of:
a)

(SEQ ID NO: 2)
TIYIGGTYIH,

b) Polypeptides that have at least 80% amino acid sequence identity with SEQ ID NO: 2,
c) Polypeptides that have 3, 2 or 1 amino acid difference with SEQ ID NO: 2,

and wherein CDR3 is chosen from the group consisting of:
a)

(SEQ ID NO: 3)
AATKWRPFISTRAAEYNY,

b) Polypeptides that have at least 80% amino acid sequence identity with SEQ ID NO: 3,
c) Polypeptides that have 3, 2 or 1 amino acid difference with SEQ ID NO: 3.

Statement 15. The method according to Statements 13 or 14, wherein the amino acid sequence of CDR1 is YTDSDYI (SEQ ID NO: 1), the amino acid sequence of CDR2 is TIYIGGTYIH (SEQ ID NO: 2), and the amino acid sequence of CDR3 is AATKWRPFISTRAAEYNY (SEQ ID NO: 3).

Statement 16. The method according to any one of Statements 13 to 15, wherein said antibody is a heavy chain variable domain derived from a heavy chain antibody (V_{HH}) or a functional fragment thereof.

Statement 17. The method according to any one of Statements 13 to 16, wherein said antibody comprises, consists essentially of or consists of an amino acid sequence having a sequence identity of at least 80%, preferably at least 90%, more preferably at least 95%, even more preferably at least 99% to SEQ ID NO: 4 or a functional fragment thereof.

Statement 18. The method according to any one of Statements 13 to 17, wherein the signal-emitting molecule comprises, consists essentially of or consist of a radionuclide.

Statement 19. The method according to any one of Statements 13 to 18, wherein the emitted signal is detected by positron emission tomography (PET) and a PET image is generated, or wherein the emitted signal is detected by single photon emission computed tomography (SPECT) and a SPECT image is generated.

Statement 20. The method according to Statement 19, further comprising a step of superimposing the PET or SPECT image with at least one computed tomography (CT) scan or at least one magnetic resonance image (MRI).

Statement 21. The method according to any one of Statements 13 to 20, wherein the subject has or is suspected of having or is under treatment for a neoplastic disease, such as a neoplastic disease as individualised in Statement 6, preferably hematological malignancy, more preferably multiple myeloma.

Statement 22. The method according to any one of Statements 13 to 21, wherein the method comprises conducting step i) on at least two distinct time points, preferably wherein a first time point is prior to the start of therapy, and a second time point is during or after therapy.

Statement 23. The method according to any one of Statements 13 to 22, further comprising detecting at least one additional signal-emitting molecule, preferably wherein said additional signal-emitting molecule is coupled to an affinity ligand capable of binding a target molecule different from CD38.

Statement 24. The method according to any one of Statements 13 to 23, wherein the subject has been administered the unlabelled anti-CD38 sdAb, followed by a second agent, which is capable of specifically binding to the anti-CD38 sdAb and which comprises the signal-emitting molecule.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The herein disclosed aspects and embodiments of the invention are further supported by the following non-limiting examples.

### EXAMPLES

### Example 1—Production and Purification of Selected Single-Domain Antibody (Nanobody®, Nb)

The generation of the anti-CD38 nanobodies 1053, 375, 551 was described in Li et al. 2016, supra, and of the anti-CD38 nanobody 2F8 in CN109232739. Briefly, the aforementioned authors immunised a Camelus bactrianus with five injections of recombinant CD38 over 4 months.

Lymphocytes were purified from the peripheral blood of the immunised animal by density centrifugation, total RNA was purified, and cDNA was generated. The DNA encoding all variable domains of heavy-chain-only antibodies generated in this animal was amplified and the amplified nanobody DNA fragments were ligated in a phage-display vector transformed into Escherichia coli TG1 cells to generate a library. Then, nanobodies were phage-displayed and biopannings were performed on recombinant CD38-conjugated beads. Interacting phages were recovered and after two additional cycles of biopanning, phages were amplified, and the binding capacity to recombinant CD38 was confirmed by ELISA. The plasmids encoding the 1053, 375, 551, and 2F8 nanobodies were obtained from the aforementioned authors.

In the present study, the DNA fragment coding for the 2F8 nanobody (henceforth, Nb2F8) was re-cloned in the expression vector pHEN6 (Arbabi Ghahroudi et al. Selection and identification of single-domain antibody fragments from camel heavy-chain antibodies. FEBS Lett. 1997, vol. 414(3), 521-6; FIG. 1A) to contain the carboxyterminal hexahistidine tail while the other three were cloned into the vector pHEN2. They were produced in 3 L of E. coli WK6 cultures each. A control nanobody cAbBcII10 was produced similarly (Saerens et al. Identification of a universal VHH framework to graft non-canonical antigen-binding loops of camel single-domain antibodies. J Mol Biol. 2005, vol. 352, 597-607). Periplasmic extracts containing the soluble nanobodies were obtained by osmotic shock. The expressed monomeric domains were purified by Immobilized Metal Affinity Chromatography and gel filtration and checked by SDS-PAGE as shown in FIG. 1B for Nb2F8. The sequence of Nb2F8 can be found in FIG. 1C.

The nanobodies were produced with an His6-tag at their C-terminus, and the 551 and 2F8 nanobodies were also produced without the His6-tag (untagged form).

Flow cytometry analysis showed the specific character of CD38-recognition by the purified nanobodies (FIG. 1D). The anti-CD38 nanobodies recognized CD38⁺ MM cell lines (e.g. RPMI-8226) and CD38⁺ Non-Hodgkin lymphoma cells, while no binding was seen with the CD38⁻ cell line or with an irrelevant nanobody (FIG. 1E).

Next, the ability of the anti-CD38 nanobodies to recognise the biotinylated CD38 antigen was investigated by Biolayer Interferometry (BLI) that allows determination of the binding kinetics parameters. The binding affinities were estimated by testing different concentrations (seven) of nanobodies with BLI. The calculated KD values of all 4 anti-CD38 nanobodies are shown in the table in FIG. 1F. These experiments were realised in a 96-well plate and consisted of different steps. First, recombinant and biotinylated CD38 (10 μg/mL) was loaded on the surface of streptavidin-coupled sensors and washed using a PBS-based buffer (PBS 50 mM pH 7, BSA 0.1%, Tween 0.02%) in order to remove any unbound CD38. Then, biocytin was added to saturate the sensors and to avoid non-specific interactions during the subsequent association step. Afterwards, the nanobodies were diluted at concentrations and brought in contact with the CD38 coated sensors to monitor the association of the nanobodies to the receptors. Finally, the sensors were placed in a fresh buffer in order to measure the dissociation rate of the nanobodies from the receptors (Kamat et al. Designing binding kinetic assay on the bio-layer interferometry (BLI) biosensor to characterize antibody-antigen interactions. Analytical Biochemistry. 2017, 536, 16-31). Octet Data Analysis software version 8.0 was used for curve fitting and the determination of the binding parameters, including the K_{D}, using a 1:1 binding model..

Nb2F8 bound to the CD38 target protein with affinities in the low (KD 0.7 nM) nanomolar range. Circular dichroism (CD) spectroscopy was performed to measure the thermal stability of the nanobodies. The Tₘ* (apparent midpoint of thermal denaturation) of Nb2F8 was 88±0.1° C. The experimentally determined characteristics of Nb2F8 and Nb551 are summarised in Table 1. CD measurements were performed in the far UV (190-250 nm) regions, using a nanobodies concentration of 0.2 mg/mL (buffer 50 mM sodium phosphate pH 7), and 0.1 cm cell pathlength. Spectra were acquired at 25° C. using a scan speed of 50 nm/min, with a 1-nm bandwidth and a 2-s integration time. The spectra were measured five times, averaged, and corrected by subtraction of the spectrum obtained with the buffer solution alone. Heat-induced unfolding transitions were monitored at 205 nm, using a similar protein concentration. The temperature was gradually increased from 25 to 97° C., at a rate of 0.5° C./min. The reversibility of this denaturation process was monitored by gradually cooling down the sample to 25° C. (Dumoulin et al. Single-domain antibody fragments with high conformational stability. Protein Science. 2002, 11, 500-515).

TABLE 1
Certain characteristics of Nb2F8 and Nb551.

| Nanobody | 2F8 | 551 |
|---|---|---|
| MW (Da) | 13800 | 15500 |
| ε (molar extinction | 2.284 | 2.034 |
| coefficient) | | |
| (L · mol⁻¹ · cm⁻¹) | | |
| Tₘ* (° C.) | 88 ± 0.1 | 72 ± 0.1 |
| K_{D} (M) | 7.3 10⁻¹⁰ ± 2.1 10⁻¹² | 8.6 10⁻¹⁰ ± 5.6 10⁻¹² |

### Example 2—In Vitro Competition with Daratumumab

To determine the competition between the anti-CD38 antibody used in clinic (daratumumab, Darzalex®) and the different nanobodies investigated herein, we performed competitive flow cytometry studies. Nb375, Nb1053, Nb551 and Nb2F8 were added and a secondary PE-labelled anti-His antibody indicated the binding of the nanobodies to RPMI-8226 cells. Staining of CD38⁺ RPMI-8226 cells with daratumumab, followed by nanobodies Nb1053 and Nb375, blocked the binding of these nanobodies to RPMI-8226 cells. In contrast, the binding of Nb2F8 and Nb551 was not blocked by previous binding of daratumumab, indicating that these antibodies bind to another epitope on the extracellular part of CD38 (FIG. 2A). The reverse experiment was also carried out to verify whether daratumumab could still bind to cells previously treated with one of the present nanobodies. There was no dissociation of the nanobodies by daratumumab binding.

In addition, the interactions between daratumumab and the present nanobodies were confirmed by an epitope binding strategy using BLI. Association of CD38 on the biosensor to the daratumumab was analysed, followed by the potential association of the nanobodies NB #2F8, NB #551, Nb #375 and Nb #1051.

There were three phases (I, II, and III) in each experiment. In phase I (loading phase), biotin-labelled recombinant CD38 protein was loaded onto the streptavidin probe on the sensor. A second binding phase with daratumumab was followed by a third binding phase with Nbs, or a second binding phase with Nbs was followed by a third binding phase with daratumumab (FIG. 2B). The Nbs 375 and 1053 could no longer bind to the CD38 receptor once the daratumumab monoclonal antibody was bound, while Nb2F8 could still bind CD38. The results for Nb551 differed according to the method used. On the basis of the BLI, upon binding Nb551 seemed to displace daratumumab from CD38. We found that after binding of daratumumab, the nanobody 2F8 was still able to bind to CD38 even after reversing the binding steps 2 and 3 (receptor+nanobody+daratumumab) (FIG. 2C).

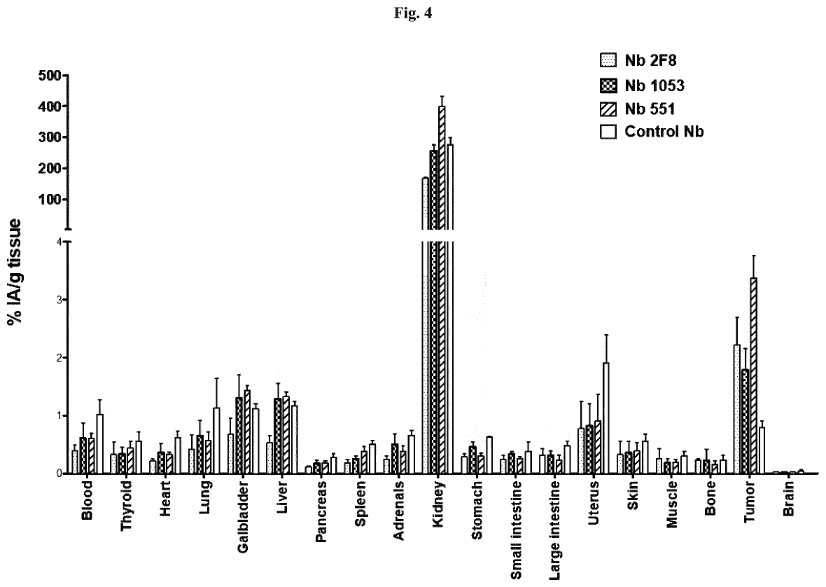

Table 2 below provides a summary of in vitro and in vivo characterisation of the different sdAbs discussed above. The affinities and competition behaviour towards the human monoclonal antibody daratumumab were obtained by the biolayer interferometry method. The sdAbs have an affinity in the nanomolar range. The data included in the biodistribution section correspond to the results obtained after radiolabelling of sdAbs with the ^{99m}Tc radioisotope (FIG. 4). The thermal stability of the proteins was evaluated by circular dichroism. NA: non applicable, protein not studied in vivo because its in vitro behaviour is similar to sdAb 1053.

| Parameters sdAbs | | #375 | #1053 | #551 | #2F8 |
|---|---|---|---|---|---|
| Affinity (K_{D}) | | 1.69 nM | 1.83 nM | 1.05 nM | 1.46 nM |
| Competition Vs | | Competitor | Competitor | Partial | Non- |
| daratumumab | | | | competitor | competitor |
| Biodistribution | Location | % IA/g (mean ± SD) | | | |
| | Tumour | NA | 1.78 ± 0.37 | 3.37 ± 0.38 | 2.22 ± 0.47 |
| | Kidneys | NA | 258.00 ± 19.08 | 402.20 ± 22.46 | 169.32 ± 4.56 |
| | Blood | NA | 0.61 ± 0.26 | 0.61 ± 0.08 | 0.39 ± 0.09 |
| | Liver | NA | 1.28 ± 0.28 | 1.33 ± 0.08 | 0.53 ± 0.12 |

| | | | | |
|---|---|---|---|---|
| Thermal stability (Tm*) | 69.1 ± 0.1° C. | 73.1 ± 0.1° C. | 72.2 ± 0.2° C. | 88.7 ± 0.3° C. |

Table 3 below provides a summary of the different binding parameters obtained after analysis by biolayer interferometry for the sdAb 2F8 conjugated to the bifunctional chelator DTPA binding to immobilised CD38 antigen using SA sensors on OctetHTX. Values are from a serial dilution of conjugate from 100 to 20 nM.

| Protein | kₒₙ (M⁻¹ s⁻¹) | k_{diss} (s⁻¹) | K_{D} (M) |
|---|---|---|---|
| 2F8-DTPA | 2.58 10⁵ ± | 4.12 10⁻⁴ ± | 1.60 10⁻⁹ ± |
| | 3.28 10² | 3.62 10⁻⁷ | 2.47 10⁻¹² |

### Example 3—^{99m}Tc-Labelling and In Vivo Biodistribution Studies of Labelled Nanobodies

Nanobodies were labelled with Technetium-99m ([^{99m}Tc(H₂O)₃(CO)₃]⁺) at their His₆-tail, as described previously (Xavier et al. Site-specific labelling of his-tagged Nanobodies with ^{99m}Tc: a practical guide. Methods Mol Biol. 2012, vol. 911, 485-90). [^{99m}Tc(H₂O)₃(CO)₃]⁺ was added to 1 mg/mL nanobody solution and incubated for 90 min at 50° C. After labelling, the ^{99m}Tc-nanobody solution was purified on a NAP-5 column to remove unbound [^{99m}Tc(H₂O)₃(CO)₃]⁺ and passed through a 0.22 m filter to eliminate possible aggregates. Further purification by gel filtration to eliminate free ^{99m}Tc-tricarbonyl resulted in a radiochemical purity >99%. Tumor-targeting potential was assessed after labelling the antibodies with ^{99m}Tc and monitoring uptake into CD38⁺ RPMI-8226 tumors in a mouse model via Single Photon Emission Computed Tomography/Computed tomography (SPECT/CT) scan and dissection analysis at 1 hour after intravenous injection. The myeloma model entails subcutaneously injecting GFP-luciferase transduced RPMI-8226 cells in NOD scid gamma mouse (NSG), bred at the animal facilities. 0.5×10⁶ cells were diluted in 100 μl Matrigel and injected 24 hours after a total-body irradiation of 2Gy. For the biodistribution studies 5 mice were included in each group, while for the therapeutic studies with ¹⁷⁷Lu labelled Nbs, 10 mice per group were included.

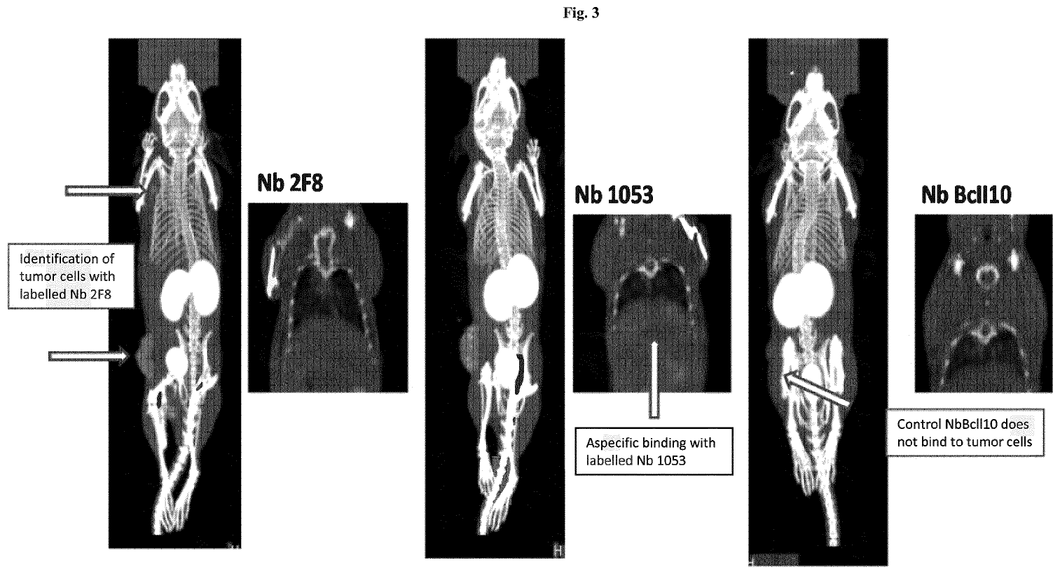

Images of SPECT/CT can be found in FIG. 3. All ^{99m}Tc-anti-CD38 Nbs showed high levels of radioactivity in kidneys and bladder. Nevertheless, all anti-CD38 Nbs showed higher tumor targeting in mouse model compared with ^{99m}Tc-NbCTRL while uptake in non-targeted tissues was low. A good ratio tumor/background was quickly obtained. A cancerous lymph node was also highlighted thanks to the high specificity of anti-CD38 Nb2F8.

Ex vivo measurements of ^{99m}Tc-nanobody uptake (biodistribution) in tumors and non-targeted organs are summarized in FIG. 4. As illustrated, ^{99m}Tc-labelled 2F8 showed a high kidney uptake, an intense activity in the bladder and low blood values. The quantitative analysis of ^{99m}Tc-nanobody uptake also showed low activities in non-targeted organs for most nanobodies. Liver accumulation varied between 0.31 and 1.59% IA/g (percentage of injected activity per gram tissue), depending on the ^{99m}Tc-nanobody. Compared to the control ^{99m}Tc-CTRL nanobody, a high tumor uptake was noted, with a tumor uptake of 2.22% IA/g and 0.79% IA/g for the Nb2F8 and NbCTRL nanobodies, respectively. Compared to other nanobodies, Nb2F8 showed less off-tumor (kidney and non-targeted tissues) binding in the different organs and thus less non-specific binding.

### Example 4—¹¹¹In-labeling of Nb2F8

Since the additional His₆-tail affects the renal uptake of the produced nanobodies (and also their biodistribution), untagged Nb2F8 was produced and conjugated to a DPTA chelator. An excess of an 2-(p-isothiocyanatobenzyl)-cyclohexyl-diethylenetriaminepentaacetic acid isomer (CHX-A″-DTPA) was conjugated for 3 hours at room temperature to the free ε-amino-groups of lysines in the nanobodies in a 0.05 M sodium carbonate buffer (pH 8.5). This reaction was quenched and the chelated nanobody was purified. The mean degree of conjugation was evaluated with ESI-Q-ToF-MS and indicated successful conjugation of the bifunctional DTPA-chelators. Radiolabelling with Indium-111 (¹¹¹In, Mallinckrodt, Petten, The Netherlands) was subsequently performed, as previously described (D'Huyvetter et al. Targeted radionuclide therapy with A 177Lu-labeled anti-HER2 nanobody. Theranostics 2014, vol. 4, 708-20). The Nb2F8-DTPA conjugate was added to metal-free 0.1 M ammonium acetate buffer pH 5.0 containing ¹¹¹In and incubated during 30 minutes. This solution was purified and filtered to eliminate possible aggregates. After radiolabelling, instant thin layer chromatography (iTLC) revealed radiochemical purity of >98%. A similar protocol was used for labelling with Lutetium-177 (¹⁷⁷Lu; ITG, Garching, Germany).

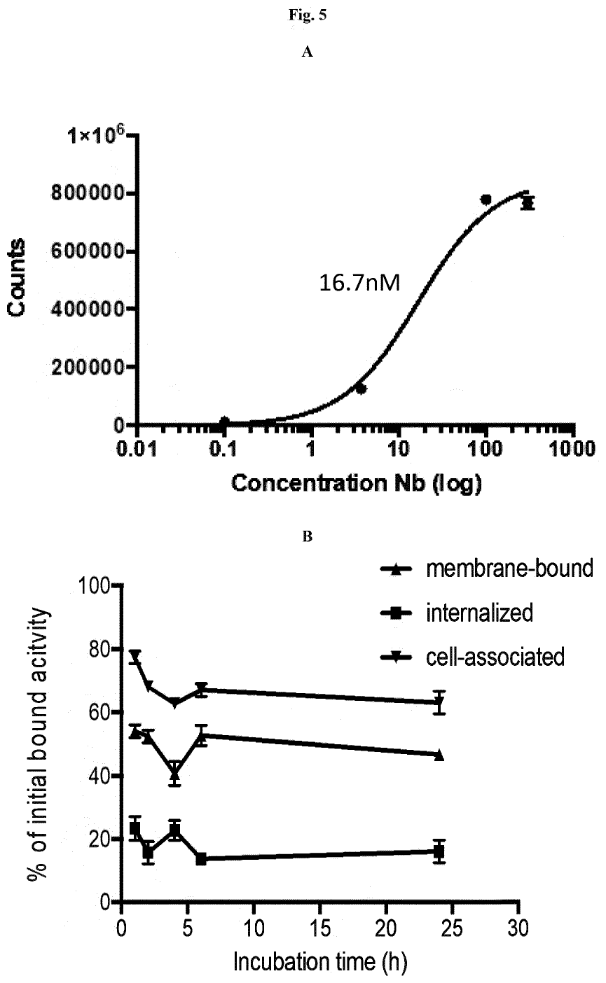

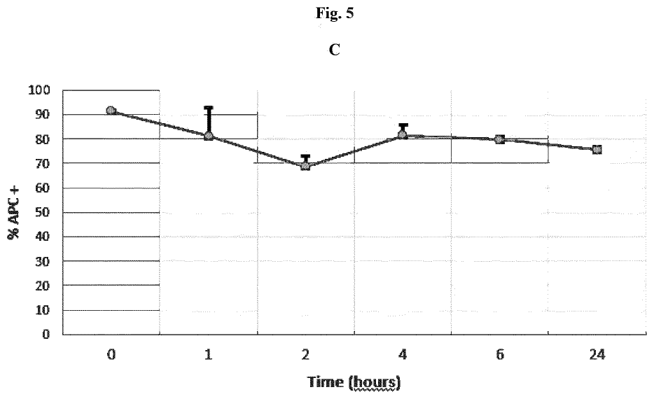

The functionality of ¹¹¹In-DTPA-Nb2F8 was verified by saturation binding experiments (using serial dilutions: 300 nM, 100 nM, 3.7 nM and 0.1 nM of the conjugated Nb) on CD38⁺ RPMI-8226 cells 1 hour at 4° C. The labelled Nb retained its functionality after labelling, since the specific binding values showed typical dose-response curves until receptor saturation, as shown in FIG. 5A. The calculated K_{D} value was 16.7 nM, which was somewhat higher than the affinity of unlabelled Nb, but still very suitable for the intended use of the labelled antibody.

### Example 6—Internalization Studies

¹¹¹In labelling allowed a longer follow-up of binding kinetics and the cellular distribution over time.

RPMI-8226 cells were cultured in tubes and were incubated with ¹¹¹In-DPTA-Nb2F8 (10 nM) for 0, 1, 2, 4, 8, 24 and 48 hours at cell culture conditions. After incubation, an acidic wash buffer (0.1 M glycine pH 2.8) was added for 6 min to remove the membrane-bound fraction of the cell-associated ¹¹¹In-DPTA-Nb2F8. Subsequently, cells were resuspended with PBS in the tubes, and the amount of membrane-bound and internalised activity was measured in a γ-counter. A minor internalisation (about 20% of the initial bound activity) was observed in the first hours and as for the percentage of membrane-bound and intracellular ¹¹¹In-DPTA-Nb2F8 remained stable during 24 hours (FIG. 5B).

In a further experiment, myeloma cells (RPMI 8226) taken from cultures were pelleted by centrifugation and resuspended with 10 nM of the nanobody 2F8 and were returned to the incubator at 37° C. for a period from 0 up to 24 hours in order to comply with the experimental conditions carried out during radioactivity tests. Six time points were achieved and each in triplicate (300.000 cells per tube). After each time point, cells were washed with PBS in order to remove all proteins not immobilised to the receptors, and labelled with the secondary antibody (APC-anti-His tag). This last incubation was carried out on ice for 20 min in order to avoid any possible additional internalisation once the incubation with the nanobody stopped. All tubes were analysed by means of a BD FACSArray Bioanalyzer System to measure the dynamics of nanobodies bound to the cell membrane. These results confirmed the conclusions from the radioactive experiments, only a minor internalisation was observed in the first time points, and membrane presence of the antibody remained stable up to 24 hours after incubation (FIG. 5C).

### Example 7—Biodistribution of ¹¹¹In and ¹⁷⁷Lu Labelled Nb2F8

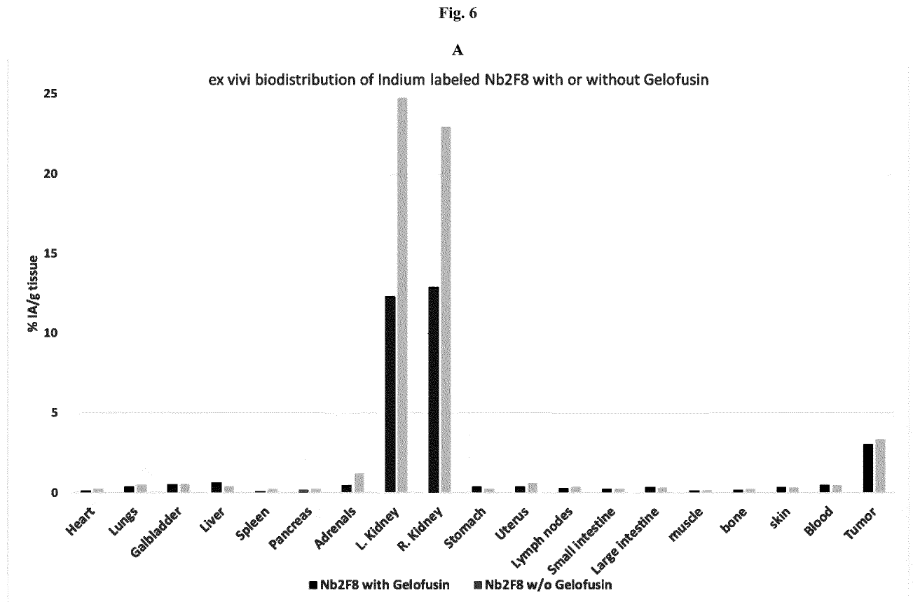

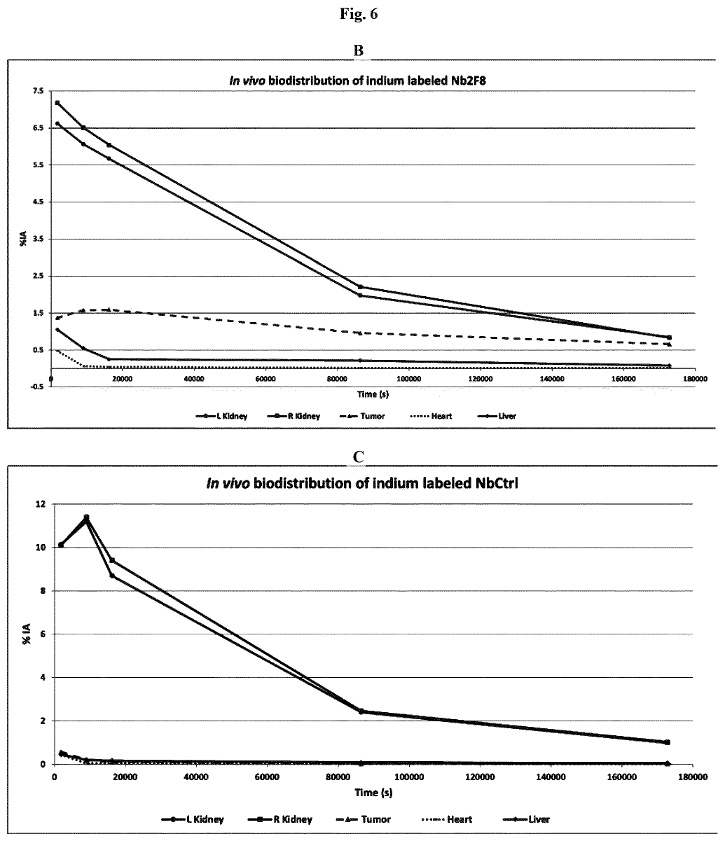

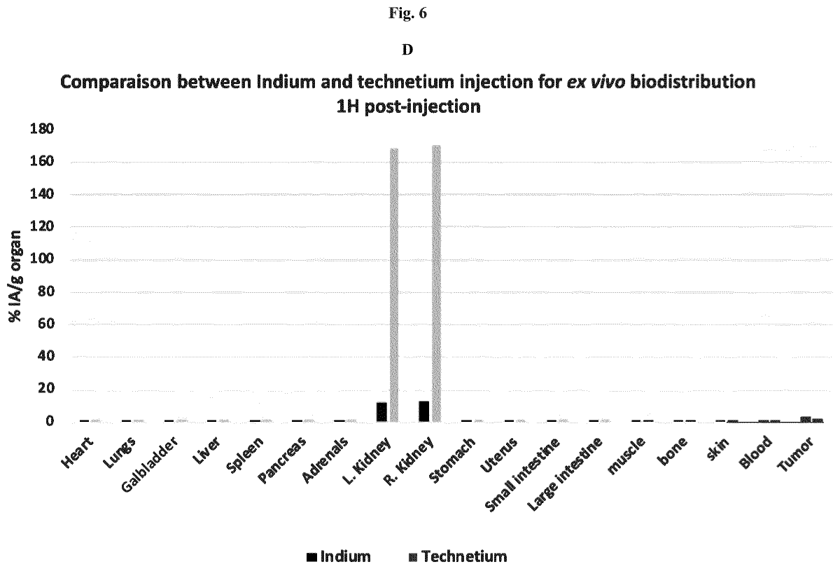

Micro-SPECT/CT images of mice bearing CD38⁺ RPMI-8226 tumors and injected with 150 μL, 0.5 mCi ¹¹¹In-DPTA-Nb2F8 showed specific tumor targeting 1 hour and at least until 48 hours after injection with a low background signal already 1 hour post-injection, except kidneys and bladder. The 1 hour profiles were similar as the ^{99m}Tc-labeled Nbs except that removing the His-Tag decreased the retention of the radiolabelled Nb in the kidneys (FIGS. 6A and 6D).

The in and ex vivo biodistribution data revealed uptake values in tumor of 3.1% IA/g and 1.4% IA/g, 1 hour and 48 hours post injection (p.i.), respectively, for ¹¹¹In-DTPA-Nb2F8 (FIGS. 6A and 6B) while ¹¹¹In-DTPA-NbCTRL noted a tumor uptake of 0.54% TA/g and 0.1% TA/g organ 1 hour and 48 hours p.i., which is significantly lower than anti-CD38 Nbs confirming the specific targeting of these Nbs (FIG. 6C).

The uptake values in the additional organs and tissues were below 0.66% IA/g, except in kidneys (FIGS. 6B and 6C). Strategies to reduce the kidneys uptake of radiolabelled protein- or peptide-based antigen-binding agents have already been investigated intensively. For example, co-infusion of the plasma-expander gelofusin could reduce renal uptake of a ¹¹¹In-labeled anti-HER2 nanobody. In our experiments, co-infusion of 150 mg/kg Gelofusine (B. Braun Medical SA, Diegem, Belgium) reduced the renal retention by at least 50% (from 23-25% IA/g to 12-13% IA/g organ) (FIG. 6A).

Hence, highest accumulation of radioactivity in the kidneys was observed for His-tagged Nbs. The lowest accumulation was associated with untagged Nbs co-infused with gelofusin with a decrease of at least 50-60% compared to Nb alone.

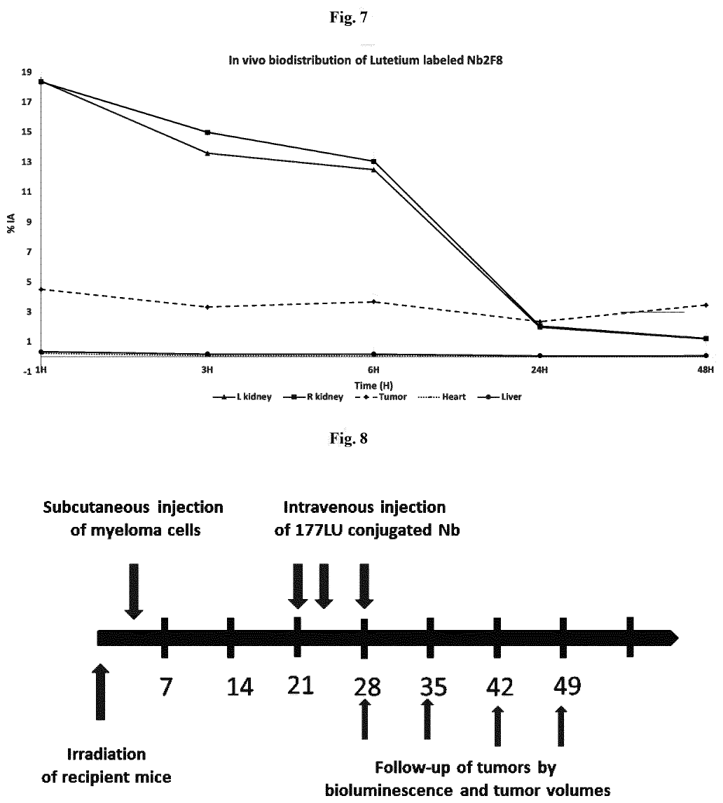

The same DTPA chelator can also be used for conjugation to ¹⁷⁷Lu. Similar biodistributions were found with ¹⁷⁷Lu-DTPA-Nb2F8. The tumor uptake values were at early time point around 4.5% IA/g organ without significant modification up to 48 H post-injection (FIG. 7). Kidneys uptake values peaked at 18% IA/g 1 hour post-injection and then decreased to 2% IA/g at 24 H p.i. and 1% IA/g at 48 H p.i. Radioactivity concentration in the other major organs and tissues was low, with values below 2% IA/g at early time points and decreasing over time.

### Example 8—Therapeutic Use of ¹⁷⁷Lu-Labelled 2F8 Nanobody in a Mouse Model

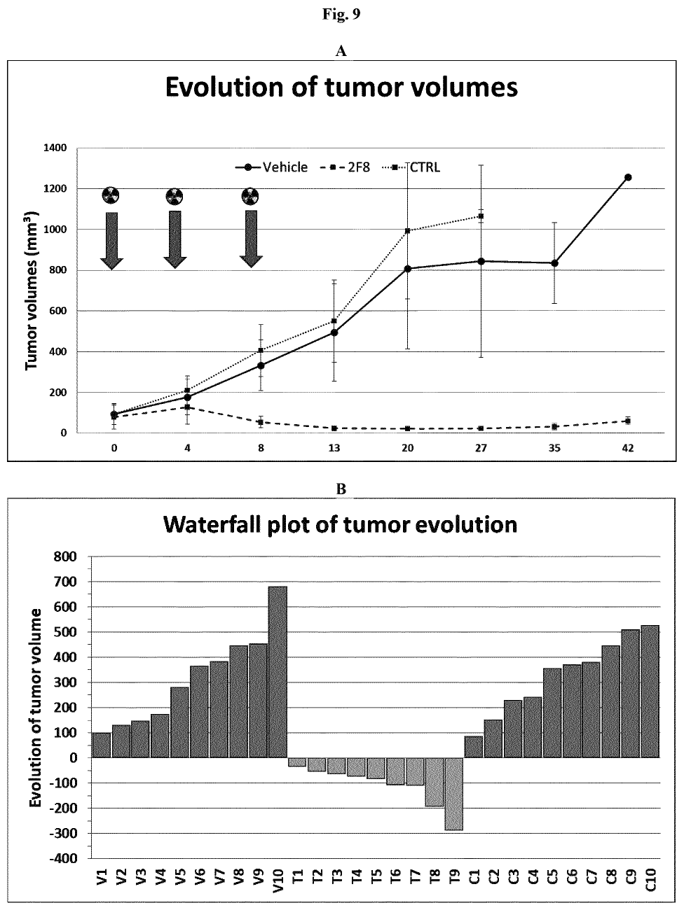

When subcutaneously injected RPMI-8226 cells became palpable (around day 20, D20), animals were randomly categorised into 3 groups (n=10). Mice in each group received 3 intravenous (i.v.) injections (D20, D24 and D27) of a phosphate buffered saline (PBS) containing either 37MBq ¹⁷⁷Lu-DTPA-Nb2F8, 37MBq ¹⁷⁷Lu-DTPA-NbCTRL, or PBS alone (FIG. 8). Animal weights were monitored weekly, as well as tumor growth through calliper measurement and bioluminescence (since RPMI-8226 cells were luciferase transduced) imaging after intraperitoneal (i.p.) injection of 150 mg/kg Luciferin. While both vehicle and ¹⁷⁷Lu-DTPA-NbCTRL showed progression of tumor masses, the tumors that received ¹⁷⁷Lu-DTPA-Nb2F8 all regressed (FIGS. 9A and 9B). FIG. 9B illustrates the evolution of the tumor volumes at day 13: all vehicle and ¹⁷⁷Lu-DTPA-NbCTRL treated mice presented an increase in tumor volume, while the tumors ¹⁷⁷Lu-DTPA-Nb2F8 treated mice regressed.

In a further experiment, therapeutic efficacy of ¹⁷⁷Lu-DTPA-2F8 was assessed in mice (10 mice per group) bearing CD38⁺ RPMI 8226 tumours. In this experiment, the number of treated groups and the radioactivity doses at each injection were different. 5 groups were defined according to the treatment regimen received according to the following strategy: 1^{st} group: 1mCi, 2^{nd} group: 750 μCi, 3^{rd} group: 500 μCi, 4^{th} group: 250 μCi, and 5^{th} group receiving only PBS as control group. 3 doses were intravenously injected at D23, D27 and D31 after inoculation of cells:

| Groups | Radioactivity | Mice | Injection |
|---|---|---|---|
| Vehicle | PBS | 10 | 3 times, 150 μL |
| Nb 2F8 | ¹⁷⁷Lu, 1 mCi | 10 | 3 times, 150 μL |
| | ¹⁷⁷Lu, 750 μCi | 10 | 3 times, 150 μL |
| | ¹⁷⁷Lu, 500 μCi | 10 | 3 times, 150 μL |
| | ¹⁷⁷Lu, 250 μCi | 10 | 3 times, 150 μL |

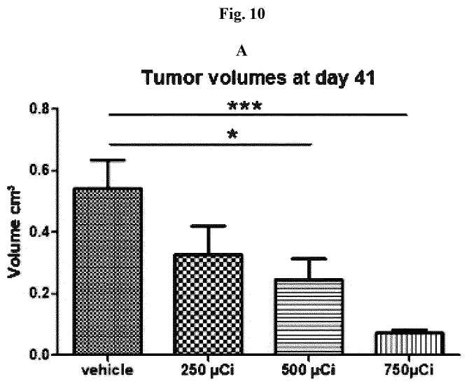

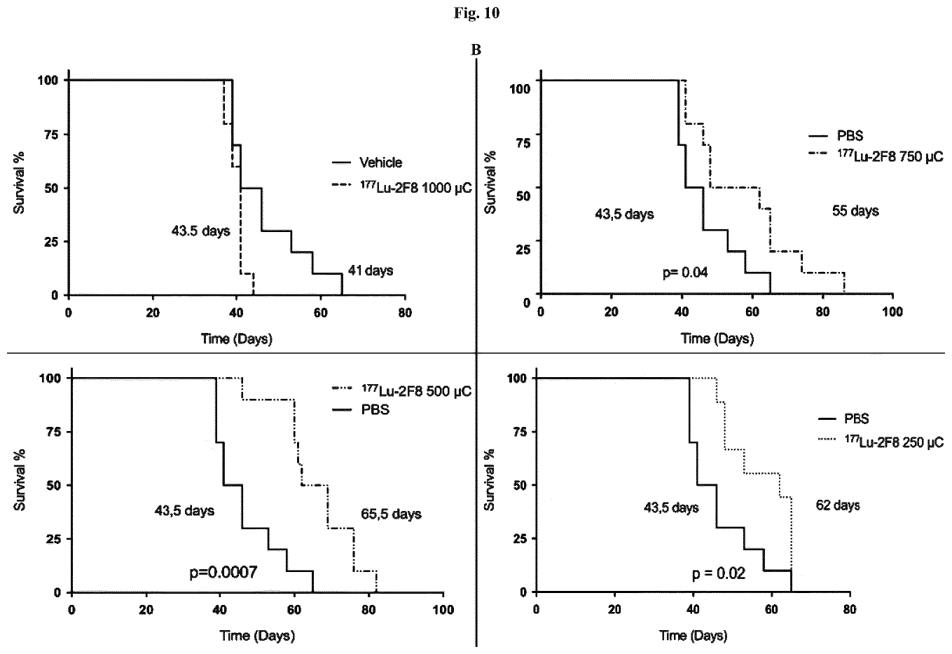

Tumour volumes were assessed daily via calliper measurements and bioluminescence imaging and mice were euthanised when tumour size exceeded 1 cm³ or with a weight loss over 15%. Different organs were recovered to analyse possible treatment-related toxicity. The direct comparison of tumour volumes measured 41 days after tumour inoculation showed a dose-dependent tumour reduction in the 250 μCi, 500 μCi, and 750 μCi dose regimens compared to vehicle solution (FIG. 10A). These changes were confirmed by similar reduction in tumour burden, quantified by bioluminescence. 2F8 was thus successfully evaluated in the framework of targeted radionuclide therapy. Repeated administration of 2F8, coupled to ¹⁷⁷Lu, resulted in a significant decrease in tumour burden and in a prolonged survival of multiple myeloma diseased mice (FIG. 10B).

### Example 9—Diagnostic/Imaging Use of ¹¹¹In-Labelled 2F8 in Human Subjects

The expression of CD38 on tumor cells, can be assessed before a certain anti-CD38 treatment is given. The obtained images allow to visualize the tumor cells and the associated expression of CD38. Administration of the anti-CD38 treatment can be realized in case of tracer accumulation in tumor sites, but should be withheld in the absence of CD38 expression. Of note, the Nb2F8 does not induce internalization and allows binding of the therapeutic monoclonal antibody daratumumab. Here for, a refractory multiple myeloma patient receives an intravenous injection of 100 MBq ¹¹¹In-DTPA-Nb2F8 nanobody formulated in NaCl 0.9% and the patient undergoes SPECT/CT one hour later. The tracer uptake is quantified by calculating the maximum standardised uptake value (SUV max) which gives an indication of the CD38 expression at certain tumor sites.

### Example 10—Theranostic Use of ¹⁷⁷Lu-Labelled 2F8 in Human Subjects

The conjugation of ¹⁷⁷Lu to the 2F8 adds a therapeutic potential to the use of radionuclide-labelled Nbs. ¹⁷⁷Lu emits gamma-particles (for diagnosis/imaging) and beta particles (for therapy). A SUBSTITUTE SHEET (RULE 26) myeloma patients receives an intravenous injection 15 MBq/kg of ¹⁷⁷Lu-labelled 2F8. 1 hour after injection, a SPECT-CT is performed to confirm binding of the labelled Nb to the tumor sites. His disease is afterwards monitored for regression of the tumor parameters and for potential haematological and renal toxicity.

### Example 11—Pre-Targeting Using 2F8

There are currently two principal radioimmunotherapy (RIT) approaches which can be contemplated: targeting of cells expressing a given antigen using labelled targeting agents (vectors), as illustrated for 2F8 in the preceding examples, or a ‘pre-targeting’ technique (Bailly et al. EJNMMI Radiopharmacy and Chemistry. 2017, vol. 2(1), 6). The latter strategy was developed in order to improve selectivity and limit the circulation of the radiolabeled agent. It is based on separation between the administration of the targeting molecule and the radiolabeled agent. After tumor uptake of the targeting agent and elimination of its free circulating form, the radiolabeled agent is injected.

Different illustrative forms of pre-targeting have been developed. One is based on the interaction between biotin and avidin or streptavidin (Yao et al. Journal of Nuclear Medicine. 1995, vol. 36(5), 837-841). Avidin is able to bind with high affinity four biotin molecules. In this approach, a specific anti-tumor antibody coupled to avidin is first injected, followed by a second injection of radiolabeled biotin. While attractive, the applicability of this technique is to some extent reduced by the immunogenicity of the avidin (Bailly et al., supra). Another pre-targeting approach involves the use of a bispecific antibody, comprising an arm directed against a tumor antigen and an arm for a radiolabeled chelator (Le Doussal et al. Cancer Research. 1990, vol. 50(11), 3445-3452). Other pre-targeting strategies have recently been introduced, based on advanced biochemistry applications, such as bio-orthogonal ligation that allows the establishment of covalent bonds between reactive groups (Rossin et al. Angewandte Chemie (International Ed. in English). 2010, vol. 49(19), 3375-3378). Another bio-engineering application integrates complementary, synthetically produced, peptide nucleic acid (PNA) oligonucleotide sequences that are conjugated to the antigen binder (or vector) and to the radionuclide (Honarvar et al. Theranostics. 2016, vol. 6(1), 93-103). The structure of PNA can be represented as shown in Honarvar:

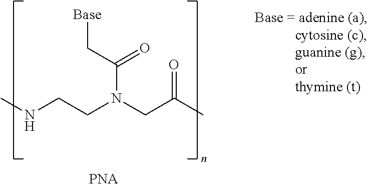

On the basis of complementarity between these conjugated sequences, the vector and the radionuclide are ligated in vivo. Advantageously, this approach is not immunogenic and causes less non-specific uptake. It employs bacterial transpeptidase (Sortase A) to conjugate the nucleotide sequences directly on the produced antibodies.

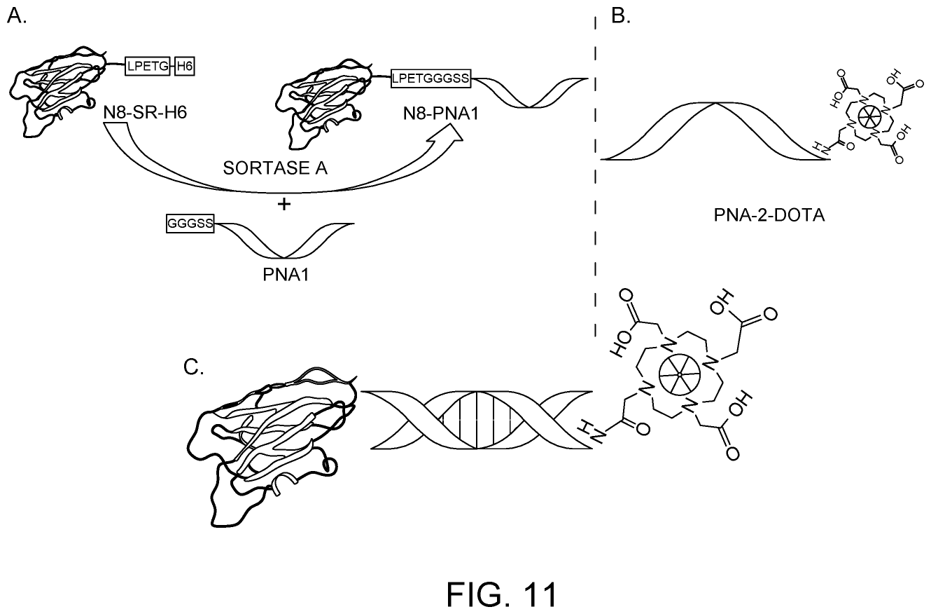

The present example describes the application of the latter pre-targeting approach to the present sdAbs, illustrated by the 2F8 nanobody (FIG. 11).

Production of Nb 2F8 with a Sortase a Recognition (SR) Motif

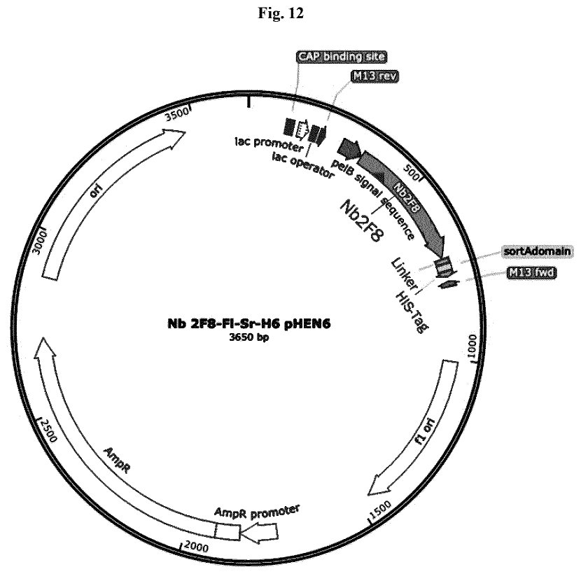

The genes encoding for Nb2F8-SR-H6 were cloned into the expression vectors pHEN6 (FIG. 12) with a special linker (FL linker; amino acid sequence EFPKSSTPPGSSGGAPGSGSGS (SEQ ID NO: 10); encoded by the nucleotide sequence GAATTTCCGAAATCGAGCACCCCGCCGGGCAGCAGCGGCGGCGCGCCG/GGCAGCGGC AGCGGCAGC (SEQ ID NO: 11)) between the nanobody sequence and the SR motif (LPETGG (SEQ ID NO: 12); encoded by the nucleotide sequence CTGCCGGAAACCGGCGGC (SEQ ID NO: 13)). This vector was transformed into the E. coli WK6 cells. Cells were cultivated in Terrific broth and protein expression was induced by the addition of isopropyl β-D-1-thiogalactopyranoside (IPTG). After pelleting the cells, the periplasmic proteins were extracted by osmotic shock in Tris-EDTA-Sucrose buffer. The fusion proteins containing a C-terminal (His)6 tag were then purified using an Immobilized Metal Affinity Chromatography on a nickel-charged agarose matrix and eluted with a linear imidazole gradient (first: 0-200 mM, and second: 200-400 mM imidazole) in buffer (HEPES 50 mM, NaCl 150 mM, imidazole 400 mM). Residual imidazole was removed by gel filtration (Sephadex G25) in 50 mM HEPES and 150 mM NaCl buffer. The presence and purity of the produced Nb2F8-FL-SR-His6 product was confirmed by SDS-PAGE.

Production of Sortase A

Sortase A was produced as previously described (Westerlund et al. Bioconjugate Chemistry. 2015, vol. 26(8), 1724-1736). Briefly, the plasmid pGBMCS-SortA provided by Addgene was subcloned into a pET-21d(+) vector and transformed into BL21-Star (DE3) E. coli cells. The Sortase enzyme becomes conjugated to hexahistidine tag that allows the same protein purification steps as described for Nb2F8-SR-H6.

Ligation of PNA1 to Nb2F8-SR-HIS

The first hybridization probe, PNA1, G-G-G-S-S-a-g-t-c-t-g-g-a-t-g-t-a-g-t-c-E-K(DOTA)-AEEA-E-NH₂ (DOTA=chelator 1,4,7,10-tetraazacyclododecane-1,4,7,10-tetraacetic acid; AEEA=linker NH₂-(PEG)₂-CH₂COOH (also referred as 2-[2-(2-aminoethoxy)ethoxy]acetic acid)) (the GGGSS part of PNA1 is denoted as SEQ ID NO: 14, the polypeptide nucleic acid part agtctggatgtagtc is denoted as SEQ ID NO: 15) was produced according to published methods (Altai et al. Methods in Molecular Biology. 2020, vol. 2105, 283-304). The ligation of Nb2F8 to PNA1 (using the Sortase A-mediated ligation) also followed the published procedures (Westerlund et al., supra).

First, the Nb2F8-SR-His fusion protein was reconstituted to the ligation buffer (HEPES 50 mM, NaCl 150 mM, CaCl₂ 10 mM, pH 7.4) and 250 nmol was put in a small microcentrifuge tube. We subsequently added 100 nmol PNA1, 285 μl ligation buffer and 20 mM NiCl₂ (to enhance the ligation yield of sortase A-mediated ligations). By adding 5 pM of Sortase A, the enzymatic reaction started and was maintained at 37° C. for 30 min on a rotating tube shaker.

For the purification of the conjugate Nb2F8-PNA1, reverse IMAC was performed using HisPur Cobalt Resins to increase the binding capacities. This cobalt resin was added to the reaction mixture at the end of the 30-min reaction time and poured into an empty column. They were eluted with a buffer HEPES 50 mM, NaCl 150 mM, pH 7.5. The different fractions were collected in 1.5 mL microcentrifuge tubes and their absorbances analyzed.

Size exclusion chromatography using Superdex 75 10/300 GL columns was performed and the recovered fractions analyzed by SDS-PAGE, by spectrophotometry on a TECAN device and sent to mass spectrometry. The presence and purity of the produced Nb2F8-PNA1 product was confirmed by SDS-PAGE.

PNA2 Conjugate with DOTA

The PNA2 conjugate with DOTA chelator was prepared according the published procedures (Westerlund et al., supra). The PNA2 conjugate was as follows: DOTA-AEEA-S-S-g-a-c-t-a-c-a-t-c-c-a-g-a-c-t-E-E-Y—NH₂ (DOTA=chelator 1,4,7,10-tetraazacyclododecane-1,4,7,10-tetraacetic acid; AEEA=linker NH₂-(PEG)₂-CH₂COOH (also referred as 2-[2-(2-aminoethoxy)ethoxy]acetic acid) (the polypeptide nucleic acid part gactacatccagact of PNA2 is denoted as SEQ ID NO: 16). The PNA2-DOTA chelator is combined with Gallium 68 radionuclide to provide PNA2-DOTA-⁶⁸Ga radiolabeled agent.

Pre-Targeting

An example of in vivo pre-targeting may be as follows. A myeloma patient receives an intravenous injection of a suitable quantity, such as 100 mg, of Nb2F8-PNA1. A certain time period after the injection, such as 30 min or 1 hour after the injection, the patient receives an intravenous injection of a suitable quantity, such as 5 mg, of PNA2-DOTA-⁶⁸Ga. His disease is afterwards monitored for regression of the tumor parameters.

1. A method for diagnosis or monitoring a neoplastic disease in a subject, the method comprising administering to the subject an anti-CD38 single-domain antibody directly or indirectly coupled to a second molecule, wherein the antibody comprises an amino acid sequence that comprises 3 complementary determining regions (CDR1 to CDR3);
wherein CDR1 is chosen from the group consisting of:
a)
(SEQ ID NO: 1)
YTDSDYI,
b) Polypeptides that have at least 80% amino acid sequence identity with SEQ ID NO: 1,
c) Polypeptides that have 3, 2 or 1 amino acid difference with SEQ ID NO: 1,
wherein CDR2 is chosen from the group consisting of:
a)
(SEQ ID NO: 2)
TIYIGGTYIH,
b) Polypeptides that have at least 80% amino acid sequence identity with SEQ ID NO: 2,
c) Polypeptides that have 3, 2 or 1 amino acid difference with SEQ ID NO: 2,
and wherein CDR3 is chosen from the group consisting of:
a)
(SEQ ID NO: 3)
AATKWRPFISTRAAEYNY,
b) Polypeptides that have at least 80% amino acid sequence identity with SEQ ID NO: 3,
c) Polypeptides that have 3, 2 or 1 amino acid difference with SEQ ID NO: 3.

2. The method according to claim 1, wherein the anti-CD38 single-domain antibody directly or indirectly coupled to the second molecule treats the neoplastic disease in the subject.

3. The method according to claim 1, wherein the amino acid sequence of CDR1 is YTDSDYI (SEQ ID NO: 1), the amino acid sequence of CDR2 is TIYIGGTYIH (SEQ ID NO: 2), and the amino acid sequence of CDR3 is
(SEQ ID NO: 3)
AATKWRPFISTRAAEYNY.

4. The method according to claim 1, wherein said antibody is a heavy chain variable domain derived from a heavy chain antibody (V_{HH}) or a functional fragment thereof.

5. The method according to claim 1, wherein said antibody comprises, consists essentially of or consists of an amino acid sequence having a sequence identity of at least 80%, preferably at least 90%, more preferably at least 95%, even more preferably at least 99% to SEQ ID NO: 4 or a functional fragment thereof:
(SEQ ID NO: 4)
QVQLVESGGGSVQAGGSLRLSCAASGYTDSDYIMAWFRQAPGKER
EVVATIYIGGTYIHYADSVKGRFTISRDNAENTVYLQMNNLKPED
TAMYYCAATKWRPFISTRAAEYNYWGQGTLVTVSS.

6. The method according to claim 1, wherein:
the neoplastic disease comprises a neoplastic cell expressing CD38 antigen at the cell surface;
the neoplastic disease is a solid tumor;
the neoplastic disease is hepatocellular carcinoma, lung cancer, melanoma, breast cancer or glioma;
the neoplastic disease is a hematological malignancy;
the neoplastic disease is multiple myeloma (MM), non-hodgkin lymphoma (NHL) or chronic lymphoid leukemia (CLL); and/or
the neoplastic disease is multiple myeloma.

7. The method according to claim 1, wherein the second molecule is detectable, or cytotoxic, or detectable and cytotoxic.

8. The method according to claim 1, wherein the second molecule is a signal-emitting molecule, preferably a signal-emitting molecule detectable by positron emission tomography (PET) or single photon emission computed tomography (SPECT), more preferably the second molecule comprises, consists essentially of or consist of a radionuclide.

9. The method according to claim 8, wherein the radionuclide is cytotoxic to cells bound by said antibody, preferably wherein the degree of toxicity is proportional to the level of CD38 expression by the cells.

10. The method according to claim 1, wherein the method further comprises treating the subject with daratumumab.

11. The method according to claim 1, wherein the method comprises administration of the unlabelled anti-CD38 sdAb, followed by administration of a second agent, which is capable of specifically binding to the anti-CD38 sdAb and which comprises the second molecule.

12. The method according to claim 1, wherein the subject has been selected as having or suspected of having the neoplastic disease, preferably a solid tumor or hematological malignancy, more preferably multiple myeloma.

13. An imaging method for evaluating or monitoring the presence, location and/or amount of CD38-expressing cells in a subject comprising the steps of:
i) detecting, in a subject to whom a detectable quantity of an anti-CD38 single-domain antibody directly or indirectly coupled to a signal-emitting molecule has been administered, signal emitted by said signal-emitting molecule coupled to said antibody; and
ii) generating an image representative of the location and/or quantity or intensity of said signal.

14. The method according to claim 13, wherein the antibody comprises an amino acid sequence that comprises 3 complementary determining regions (CDR1 to CDR3);
wherein CDR1 is chosen from the group consisting of:
a)
(SEQ ID NO: 1)
YTDSDYI,
b) Polypeptides that have at least 80% amino acid sequence identity with SEQ ID NO: 1,
c) Polypeptides that have 3, 2 or 1 amino acid difference with SEQ ID NO: 1,
wherein CDR2 is chosen from the group consisting of:
a)
(SEQ ID NO: 2)
TIYIGGTYIH,
b) Polypeptides that have at least 80% amino acid sequence identity with SEQ ID NO: 2,
c) Polypeptides that have 3, 2 or 1 amino acid difference with SEQ ID NO: 2,
and wherein CDR3 is chosen from the group consisting of:
a)
(SEQ ID NO: 3)
AATKWRPFISTRAAEYNY,
b) Polypeptides that have at least 80% amino acid sequence identity with SEQ ID NO: 3,
c) Polypeptides that have 3, 2 or 1 amino acid difference with SEQ ID NO: 3.

15. The method according to claim 14, wherein the amino acid sequence of CDR1 is YTDSDYI (SEQ ID NO: 1), the amino acid sequence of CDR2 is TIYIGGTYIH (SEQ ID NO: 2), and the amino acid sequence of CDR3 is
(SEQ ID NO: 3)
AATKWRPFISTRAAEYNY.

16. The method according to claim 13, wherein:
said antibody is a heavy chain variable domain derived from a heavy chain antibody (V_{HH}) or a functional fragment thereof; and/or
said antibody comprises, consists essentially of or consists of an amino acid sequence having a sequence identity of at least 80%, preferably at least 90%, more preferably at least 95%, even more preferably at least 99% to SEQ ID NO: 4 or a functional fragment thereof.

17. The method according to claim 13, wherein the signal-emitting molecule comprises, consists essentially of or consist of a radionuclide.

18. The method according to claim 13, wherein the emitted signal is detected by positron emission tomography (PET) and a PET image is generated, or wherein the emitted signal is detected by single photon emission computed tomography (SPECT) and a SPECT image is generated.

19. The method according to claim 18, further comprising a step of superimposing the PET or SPECT image with at least one computed tomography (CT) scan or at least one magnetic resonance image (MRI).

20. The method according to claim 13, wherein the subject has or is suspected of having or is under treatment for a neoplastic disease, preferably a solid tumor or a hematological malignancy, more preferably multiple myeloma.

21. The method according to claim 13, wherein the method comprises conducting step i) on at least two distinct time points, preferably wherein a first time point is prior to the start of therapy, and a second time point is during or after therapy.

22. The method according to claim 13, further comprising detecting at least one additional signal-emitting molecule, preferably wherein said additional signal-emitting molecule is coupled to an affinity ligand capable of binding a target molecule different from CD38.

23. The method according to claim 13, wherein the subject has been administered the unlabelled anti-CD38 sdAb, followed by a second agent, which is capable of specifically binding to the anti-CD38 sdAb and which comprises the signal-emitting molecule.

24. The method according to claim 2, wherein the method further comprises treating the subject with daratumumab.
